(19) United States
(12) Patent Application Publication
(10) Pub. No.: US 2024/0084258 A1
(43) Pub. Date: Mar. 14, 2024

(54) METHOD FOR PREPARING BROWN ADIPOCYTE

(71) Applicant: KYOTO PREFECTURAL PUBLIC UNIVERSITY CORPORATION, Kyoto (JP)

(72) Inventors: Kenta YAMAMOTO, Kyoto-shi (JP); Tsunao Kishida, Kyoto-shi (JP); Toshiro Yamamoto, Kyoto-shi (JP); Osam Mazda, Kyoto-shi (JP)

(73) Assignee: KYOTO PREFECTURAL PUBLIC UNIVERSITY CORPORATION, Kyoto (JP)

(21) Appl. No.: 18/517,402

(22) Filed: Nov. 22, 2023

Related U.S. Application Data
(63) Continuation of application No. 15/750,285, filed on Jul. 3, 2018.

(30) Foreign Application Priority Data
Aug. 7, 2015 (JP) 2015-157697

(51) Int. Cl. C12N 5/077; A61K 35/35

(52) U.S. Cl. CPC C12N 5/0653; A61K 35/35; C12N 2501/01; C12N 2501/15; C12N 2501/30; C12N 2501/727; C12N 2501/999; C12N 2506/1307

(57) ABSTRACT

The present invention aims to provide a brown adipocyte and a generation method thereof, a transplantation material containing a brown adipocyte, a prophylactic agent or therapeutic agent containing a brown adipocyte for various diseases and conditions, and use thereof. Provided is a method for generating a brown adipocyte, including converting a differentiated somatic cell of a mammal to a brown adipocyte by culturing the somatic cell in a medium in the presence of at least one kind of a compound selected from the group consisting of (1) a TGFβ/SMAD pathway inhibitor, (2) a casein kinase 1 inhibitor, (3) a cAMP inducer and (4) a MEK/ERK pathway inhibitor.

### CROSS-REFERENCE TO THE RELATED APPLICATION

The present invention claims priority to Japanese Patent Application No. 2015-157697 filed on Aug. 7, 2015, which is incorporated herein by reference in their entireties.

### TECHNICAL FIELD

The present invention relates to a brown adipocyte and a generating method thereof. The present invention also relates to a prophylactic or therapeutic agent for obesity, diabetes, impaired glucose tolerance, lipid metabolism abnormality, arteriosclerotic disease, hypertension, hyperuricemia, gout, non-alcoholic steatohepatitis or metabolic syndrome and use thereof.

### BACKGROUND ART

Obesity and metabolic diseases related thereto, for example, diabetes, metabolic syndrome and the like are extremely important medical and social problems in industrialized countries. In obesity, white adipocytes not only store excess energy derived from food as fatty acids but also produce various hormones and cytokines to induce impaired glucose tolerance and lipid metabolism abnormality, which in turn causes type II diabetes, arteriosclerotic diseases, hypertension, hyperuricemia, gout, non-alcoholic steatohepatitis and the like.

On the other hand, brown fat (Brown Adipose; BA; cells oxidatively decompose fatty acids and release energy thereof as heat, opposite to white adipocytes. This occurs since an inner mitochondrial membrane protein, UCP1 (Uncoupling protein 1), specifically expressed by BA cells uncouples oxidative phosphorylation. In rodents such as mouse and the like, BA cells are present in the interscapulum, posterior region of neck, mediastinum, perirenal region and the like. In addition, BA cell is known to suppress obesity and impaired glucose tolerance from the UCP1 knockout mouse analysis etc.

Brown adipocytes have been considered to exist only in infancy and not in adults in human. However, it was clarified in 2009 that brown adipocytes are also present in adults in the supraclavicular subcutaneous tissue, periaorta and the like (non-patent documents 1-3). The number and function of brown adipocytes show large individual differences, and they are inversely correlated with BMI (Body Mass Index) and fasting blood glucose. They are numerous in lean type human and extremely low in patients with obesity, diabetes or hyperlipidemia. Therefore, brown adipocytes have important significance in analyzing genetic predisposition, searching for environmental factors, elucidating pathology, or developing techniques for new diagnosis method, judgment of treatment effect and the like, each relating to diseases such as obesity, diabetes, hyperlipidemia and the like. Brown adipocytes are also considered to be extremely beneficial for the development of new therapeutic drugs for these diseases. Furthermore, if supplementation of brown adipocytes in patients with obesity, diabetes, hyperlipidemia, metabolic syndrome or the like is possible, it may be a new therapeutic approach for these diseases.

While a method for obtaining mesenchymal stem cells, and then brown adipocytes, from human iPS cells is known (non-so patent documents 4, 5), when brown adipocytes are induced from fibroblasts via iPS cell, it takes time to obtain the final adipocytes and it is difficult to deny the risk of oncogenesi when the obtained cells are transplanted.

For example, the following reports are available regarding direct conversion of somatic cells:
mouse fibroblast→chondrocyte (SOX9+Klf4+c-Myc genes were introduced)
mouse fibroblast→cardiac muscle cell (GATA4+Mef2c+Tbx5 genes were introduced)
mouse fibroblast→liver cell (Hnf4α+(Foxa1, Foxa2 or Foxa3) genes were introduced
mouse fibroblast→neural stem cell (Sox2+FoxG1 genes were introduced, and the like),
mouse or human cell→hematopoietic stem cell.

It has heretofore been known to transfect PRDM16 and C/EBPβ into myoblast or fibroblast, and induce same to “brown adipocyte-like cell” (patent document 2 and non-patent document 6). However, the cells induced with PRDM16 and C/EBPβ show a very low UCP1 expression level and the like, and only show insufficient properties as brown adipocytes.

Patent document 3 discloses a technique for inducing highly functional brown adipocytes by introducing C/EBP-β and c-Myc gene into human fibroblasts (patent document 3). In patent document 3, when brown adipocytes were induced from mouse fibroblasts and transplanted to diabetic mouse, impaired glucose tolerance, insulin resistance, dyslipidemia and body weight increase could be remarkably suppressed. Furthermore, when brown adipocytes were induced from mouse fibroblasts and transplanted to syngeneic mouse and a high-fat diet was given, diet-induced obesity, impaired glucose tolerance, insulin resistance and dyslipidemia could be suppressed nearly completely (to the same level as mouse fed a normal diet) (patent document 3).

In such technique for inducing brown adipocytes by introducing a gene, however, it was not easy to deny the risk of canceration and the like of the cells transplanted after transplantation of the obtained brown adipocytes. In addition, the induction technique is complicated and high expenses are necessary to ensure safety and verification.

If a technique for converting a differentiated somatic cell to a brown adipocyte can be provided without gene transfer, a regenerative medicine for diabetes, obesity, metabolic syndrome and the like, which is safe, economical and highly useful, may be provided. Using the obtained brown adipocytes, the development of a drug for these diseases, which is based on a new action mechanism, and the like are expected.

### DOCUMENT LIST

### Patent Documents

patent document 1: WO 2010/071210
patent document 2: WO 2010/080985A8
patent document 3: WO 2014010746 A1

### Non-Patent Documents

non-patent document 1: Saito M. et al., Diabetes 58:1526, 2009
non-patent document 2: Cypess A. M. et al., N Eng J Med 360: 1509, 2009
non-patent document 3: Van Merken Lichtenbelt W. D. et al., N Engl J Med 360: 1500, 2009
non-patent document 4: Tim Ahfeldt et al., Nature Cell Biology Vol. 14, No. 2, 2012
non-patent document 5: Nishio et al., Cell Metabolism, 16, 394, 2012
non-patent document 6: Kajimura S, et al. Nature 460: 1154, 2009
non-patent document 7: Callahan J F, et al., J Med Chem 45: 999, 2002

### SUMMARY OF THE INVENTION

### Problems to be Solved by the Invention

The present invention provides a brown adipocyte and a generating method thereof, a transplantation material containing a brown adipocyte, a prophylactic agent or therapeutic agent containing a brown adipocyte for various diseases and conditions, and use thereof.

The present invention also aims to provide a prophylactic or therapeutic agent for or a method for the prophylaxis or treatment of obesity, diabetes, impaired glucose tolerance, lipid metabolism abnormality, arteriosclerotic disease, hypertension, hyperuricemia, gout, non-alcoholic steatohepatitis or metabolic syndrome, a transplantation material effective for the prophylaxis or treatment of the diseases or conditions and a preparation method thereof.

To be specific, the present invention aims to provide a technique for converting a somatic cell to a brown adipocyte without gene transfer.

### Means of Solving the Problems

The present inventor has found that a differentiated somatic cell of a mammal can be converted to a brown adipocyte by culturing the aforementioned somatic cell in a medium in the presence of at least one kind of a compound selected from the group consisting of (1) a TGFβ/SMAD pathway inhibitor, (2) a casein kinase 1 inhibitor, (3) a cAMP inducer and (4) a MEK/ERK pathway inhibitor.

No report exists that teaches induction of a somatic cell to a brown adipocyte by using these compounds.

The present invention encompasses the following invention.
Item 1: a method for generating a brown adipocyte, comprising converting a differentiated somatic cell of a mammal to a brown adipocyte by culturing the aforementioned somatic cell in a medium in the presence of at least one kind of a compound selected from the group consisting of
(1) a TGFβ/SMAD pathway inhibitor,
(2) a casein kinase 1 inhibitor,
(3) a cAMP inducer, and
(4) a MEK/ERK pathway inhibitor.
Item 2: the method of item 1, wherein the aforementioned somatic cell is fibroblast.
Item 3: the method of item 1 or 2, wherein the aforementioned medium is an adipocyte induction medium added with a thyroid hormone and a PPARγ agonist.
Item 4: an inducer for converting a differentiated somatic cell to a brown adipocyte, comprising at least one kind of a compound selected from the group consisting of
(1) a TGFβ/SMAD pathway inhibitor,
(2) a casein kinase 1 inhibitor,
(3) a cAMP inducer, and
(4) a MEK/ERK pathway inhibitor.
Item 5: a kit for converting a differentiated somatic cell to a brown adipocyte, comprising at least one kind of a compound selected from the group consisting of
(1) a TGFβ/SMAD pathway inhibitor,
(2) a casein kinase 1 inhibitor,
(3) a cAMP inducer, and
(4) a MEK/ERK pathway inhibitor, and
a medium.
Item 6: the kit of item 5, wherein the aforementioned medium is an adipocyte induction medium added with a thyroid hormone and a PPARγ agonist.
Item 7: a prophylactic or therapeutic agent for obesity, diabetes, impaired glucose tolerance, lipid metabolism abnormality, arteriosclerotic disease, hypertension, hyperuricemia, gout, non-alcoholic steatohepatitis or metabolic syndrome, comprising a brown adipocyte generated by the method of any one of items 1 to 3 as an active ingredient.
Item 8: use of a brown adipocyte generated by the method of any one of items 1 to 3 in the prophylaxis or treatment of obesity, diabetes, impaired glucose tolerance, lipid metabolism abnormality, arteriosclerotic disease, hypertension, hyperuricemia, gout, non-alcoholic steatohepatitis or metabolic syndrome.
Item 9: a transplantation material comprising a brown adipocyte generated by the method of any one of items 1 to 8.

### Effect of the Invention

In the present invention, brown adipocytes can be provided from somatic cells differentiated in a short time by the action of a low-molecular-weight compound. Brown adipocytes can be easily induced from the somatic cells of a transplantation recipient and therefore, problems of immunological rejection and the like do not occur even when s brown adipocyte or a bone tissue produced therefrom is transplanted. In addition, problems caused by pluripotent stem cells such as canceration and the like can be avoided because brown adipocytes can be directly induced from somatic cells without intervention of iPS cell or ES cell. On the other hand, it is also possible to produce the cells in advance to storage in a bank and use the cells therefrom for allotransplantation or xenotransplantation to patients.

### BRIEF DESCRIPTION OF THE DRAWINGS

FIG. 1 shows phase contrast microscopic images of cells stained with Oil Red O. magnification ×100

FIG. 2 is a graph showing mRNA expression of UCP-1 gene as quantified by real time RT-PCR after extraction of RNA from cells.

FIG. 3 is a graph showing mRNA expression of UCP-1 gene as quantified by real time RT-PCR after extraction of RNA from cells.

FIG. 4 is a graph showing mRNA expression of CIDEA gene as quantified by real time RT-PCR after extraction of RNA from cells.

FIG. 5 is a graph showing mRNA expression of PGC-1α gene as quantified by real time RT-PCR after extraction of RNA from cells.

FIG. 6 is a graph showing mRNA expression of AdipoQ gene as quantified by real time RT-PCR after extraction of RNA from cells.

FIG. 7A shows fluorescence microscopic images of cells with fat droplet stained with BODIPY. magnification ×200

FIG. 7B is a graph showing fluorescence intensity of BODIPY staining in FIG. 7A.

FIG. 7C shows black-and-white inverted view of the BODIPY-stained images of FIG. 7A.

FIG. 8A shows fluorescence microscopic images of cells immunostained with UCP1. magnification ×200

FIG. 8B is a graph showing fluorescence intensity of UCP-1 staining in FIG. 8A.

FIG. 8C shows black-and-white inverted view of the UCP-1-stained images of FIG. 8A.

FIG. 9 shows microscopic images of cells stained with Oil Red O. magnification ×100

FIG. 10A shows fluorescence microscopic images of cells in which fat droplet was stained with BODIPY and immunostained with UCP1, and the nucleus was stained with DAPI. magnification ×200

FIG. 10B shows black-and-white inverted view of the stained images of FIG. 10A.

FIG. 11 schematically shows the outline of TGFβ/SMAD pathway.

FIG. 12 schematically shows the outline of MEK/ERK pathway.

FIG. 13 is a graph showing mRNA expression of UCP-1 gene as quantified by real time RT-PCR after extraction of RNA from cells.

FIG. 14A shows fluorescence microscopic images of cells immunostained with UCP1. magnification ×100

FIG. 14B shows black-and-white inverted view of the UCP-1-stained images of FIG. 14A.

FIG. 15 is a graph showing mRNA expression of UCP-1 gene as quantified by real time RT-PCR after extraction of RNA from cells.

FIG. 16A shows fluorescence microscopic images of cells immunostained with UCP1. magnification ×100 FIG. 16B shows black-and-white inverted view of the UCP-1-stained images of FIG. 16A.

FIG. 17 is a graph showing mRNA expression of CIDEA gene and KCNK3 gene as quantified by real time RT-PCR after extraction of RNA from cells.

### DESCRIPTION OF EMBODIMENTS

The present invention relates to a method for converting a differentiated somatic cell of a mammal to a brown adipocyte. The method enables generation of a brown adipocyte by using somatic cell as a starting material. The “converting” means to change a somatic cell to a brown adipocyte of interest. One of the preferable embodiments of the method of the present invention is a method for converting a somatic cell to a brown adipocyte without going through a step of reprogramming a cell, which is represented by the production of iPS cell, also called “direct reprogramming”, “direct conversion”.

### Brown Adipocyte

The present invention provides a method for adjusting a brown adipocyte. The brown adipocyte is one of the two types of adipocyte present together with white adipocyte in mammals. As a cell having a shape and function similar to those of brown adipocyte, cells called Beige cell or Brite cell are also known, and such cells are also encompassed in the “brown adipocyte” in the present specification.

The presence of a brown adipocyte can be confirmed by a known method. For example, staining with fluorescence dye capable of detecting fat droplet in the cell, and detection of a gene product (mRNA or protein) expressed in brown adipocyte can be mentioned. As a fluorescence dye capable of detecting fat droplet in the cell, Oil Red O, BODIPY and the like can be mentioned. As gene products expressed in brown adipocytes, UCP-1, CIDEA, PCG-1α, DIO2, Cox8b, Otop, AdipoQ and the like can be mentioned. Among these, UCP-1 (Uncoupling protein 1) is a gene specifically expressed in brown adipocytes, and is considered to encode a protein in the inner membrane of mitochondria that uncouples oxidative phosphorylation and is the basis for the function of brown adipocyte. Thus, it is one of the particularly preferable indices of brown adipocytes.

### Somatic Cell

The differentiated somatic cell of a mammal to be the target of the method of the present invention is not particularly limited as long as it is derived from a mammal and is not a brown adipocyte itself or a cell having an ability to differentiate into brown adipocyte in the body.

Examples of the kind of the somatic cell include fibroblast, epithelial cell (skin epidermal cell, mouth cavity mucosal epithelial cell, airway mucosal epithelial cell, intestinal mucosal epithelial cell and the like), epidermal cell, gingiva cell (gingiva fibroblast, gingiva epithelial cell), pulp cell, white adipocyte, subcutaneous fat, visceral fat, muscle, blood cell and the like, with preference given to fibroblast, gingiva cell, mouth cavity mucosal epithelial cell, pulp cell, adipocyte, epidermal cell (keratinocyte), blood cell and the like.

In addition, somatic cells produced by inducing differentiation of or dedifferentiating or reprogramming somatic stem cells such as mesenchymal stem cell (MSC), neural stem cell, hepatic stem cell, intestinal stem cell, skin stem cell, hair follicle stem cell, pigment cell stem cell and the like can also be mentioned. In addition, different somatic cells produced by inducing differentiation of or dedifferentiating or reprogramming various somatic cells can also be mentioned. In addition, somatic cells produced by inducing differentiation of or dedifferentiating or reprogramming germline cells can also be mentioned germline cells can also be mentioned.

In addition, somatic cells produced by inducing differentiation of or reprogramming embryonic stem cells (ES cells) or induced pluripotent stem cells (iPS cells) can also be mentioned.

Although not strictly somatic cells, ES cell, iPS cell and germline cell are also encompassed in the “somatic cell” of the present invention (in this case, “somatic cell” is referred to as “ES cell”, “iPS cell” or “germline cell”).

Cultured cells are also recited and somatic cells induced by differentiation induction or dedifferentiation or reprogramming of cultured cells can also be mentioned.

Examples of the mammal include mouse, rat, hamster, human, dog, cat, monkey, rabbit, bovine, horse, swine and the like. Somatic cell is particularly preferably derived from human. The age of the individual from which the somatic cell is derived is not limited, and the individual may be adult, infant or fetal. In the present specification, cells derived from fetus and cells derived from placenta, amniotic membrane, umbilical cord and the like are also encompassed in the “somatic cell”.

When generated brown adipocytes are transplanted to the body, somatic cells derived from the test subject who receives transplantation (autologous cells) are preferably used to reduce the risk of infection, rejection and the like. However, instead of autologous cells, brown adipocytes produced from somatic cells of other people or other animal can be used for transplantation. Alternatively, brown adipocytes may be produced from somatic cells produced in advance from other people or other animals and used for transplantation. Alternatively, brown adipocytes produced in advance from somatic cells of other people or other animals can be used for transplantation. That is, a brown adipocyte bank or a bank of brown adipocyte progenitor cells may be produced and used for transplantation purposes. In this case, to reduce the risk of rejection response and the like, blood type and MHC can be typed in advance. In addition, it is possible to confirm in advance the characters, tumorigenicity and the like of the brown adipocytes.

### Medium

The medium to be used in the method of the present invention is not particularly limited. General liquid media such as DMEM (Dulbecco's Modified Eagle's Medium), EMEM (Eagle's minimal essential medium) and the like can be used. Where necessary, components such as serum component (Fetal Bovine Serum (FBS), Human Serum (Serum)), antibiotics such as streptomycin, penicillin and the like and Non-Essential Amino Acid and the like can be added.

In view of the high generation efficiency of brown adipocytes, it is preferable to use, as a medium, a differentiation induction medium for differentiating adipocytes. The “differentiation induction medium for differentiating adipocytes” refers to a medium containing components capable of differentiating pluripotent stem cells (ES cell, iPS cell and the like) into adipocytes. As the differentiation induction medium, the above-mentioned general liquid medium (optionally added with components where necessary) added with the following components (one or more kinds) can be mentioned:

insulin (Insulin) (e.g., concentration about 0.01-100 μg/mL, more preferably about 0.1-10 μg/mL); 3-isobutyl-1-methylxanthine (IBMX) (e.g., concentration about 0.01-100 mM, more preferably about 0.1-10 mM); dexamethasone (Dexametazone) (e.g., concentration about 0.01-100 μM, more preferably about 0.1-10 μM). In addition, indomethacin (Indometacin) (e.g., concentration about 0.001-10 mM, more preferably about 0.01-1 mM) may be added.

Specific examples of adipocyte induction medium include, but are not limited to, 10% FBS-added DMEM+MDI medium (10% FBS-added DMEM containing 0.5 mM isobutylmethylxanthine (IBMX), 0.5 μM dexametazone and 1 μg/mL Insulin).

In view of the high conversion efficiency to brown adipocyte, it is preferable to further add a thyroid hormone such as triiodothyronine (Triiodothyronine,T3), thyroxine (Thyroxine, T4) and the like (e.g., concentration about 0.01-100 nM, more preferably about 0.1-10 nM) or Peroxisome Proliferator-Activated Receptor-γ (PPAR-γ) agonist (e.g., concentration about 0.01-100 μM, more preferably about 0.1-10 μM), more preferably the both, to the adipocyte induction medium.

Examples of the PPAR-γ agonist include thiazolidinedione compounds such as Rosiglitazone, Ciglitazone, GW1929, nTZDpa, Pioglitazone Hydrochloride, Troglitazone and the like.

A preferable embodiment of the medium for inducing brown adipocyte includes (1) DMEM medium added with FBS 10%, 0.5 mM IBMX, 125 nM Indomethacin, 1 microM Dexamethasone, 850 nM insulin, thyroid hormone such as triiodothyronine (Triiodothyronine, T3), thyroxine (Thyroxine, T4) and the like (e.g., concentration about 0.01-100 nM, more preferably about 0.1-10 nM) and 1 μM Rosiglitazone, and [2] DMEM medium added with 10% FBS, 850 nM insulin, 1 nM T3, Peroxisome Proliferator-Activated Receptor-γ (PPAR-γ) agonist (e.g., concentration about 0.01-100 μM, more preferably about 0.1-10 μM). It is particularly desirable to use [1] on day 1-day 2 and [2] on day 3 and thereafter, though the use is not limited thereto.

### Compound

In the method of the present invention, a differentiated somatic cell of a mammal is cultured in a medium in the presence of at least one kind of a compound selected from the group consisting of
(1) a TGFβ/SMAD pathway inhibitor,
(2) a casein kinase 1 inhibitor,
(3) a cAMP inducer, and
(4) a MEK/ERK pathway inhibitor. Each compound is explained below.

### TGF-β/SMAD Pathway Inhibitor

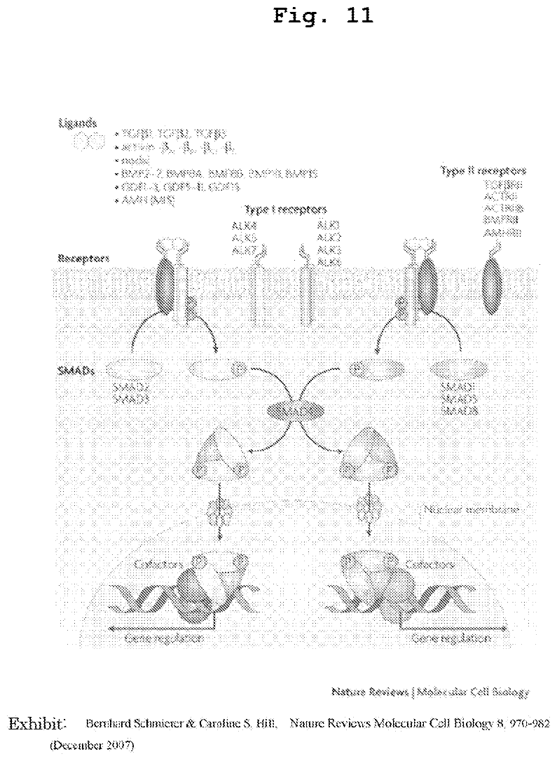

TGF-β/SMAD pathway inhibitor means a compound capable of inhibiting the activity of protein belonging to the TGF-β/SMAD pathway. The TGF-β/SMAD pathway is a signal pathway known to those of ordinary skill in the art and is schematically shown in FIG. 11.

The TGF-β/SMAD pathway is mainly constituted of a ligand constituted of protein belonging to the TGF-β superfamily (TGF-β1, TGF-β2, TGF-β3, activin-βA, activin-βB, activin-βC, activin-βϵ, noda1, BMP2, BMP3, BMP4, BMP5, BMP6, BMP7, BMP8A, BMP8B, BMP10, BMP15, GDF1, GDF2, GDF3, GDF5, GDF6, GDF7, GDF8, GDF9, GDF10, GDF11, GDF15, AMH (MIS) and the like), protein belonging to the TGF-β type I receptor family and protein belonging to the TGF-β type II receptor family constituting heterodimeric receptors, and protein belonging to the SMAD family and is an intracellular signal molecule (effector) (particularly SMAD2, SMAD3, SMAD4, SMAD1, SMAD5 or SMAD8).

In the TGF-β/SMAD pathway, when the ligand binds to a dimeric receptor, TGF-β type I receptor protein, which is a kinase type receptor, phosphorylates the SMAD protein and transmits a signal downstream. In the present specification, therefore, a molecule that suppresses any of the cytokine of TGF-β superfamily and the proteins of TGF-β type I receptor family, TGF-β type II receptor family and SMAD family (particularly SMAD2, SMAD3, SMAD4, SMAD1, SMAD5 or SMAD8) is called a TGF-β/SMAD pathway inhibitor.

The “TGF-β/SMAD pathway inhibitor” encompasses not only low-molecular-weight compounds which are inhibitors in the narrow sense but also receptor antagonist; soluble receptor; antibody, aptamer and peptide that bind to a protein of a pathway and have an activity to inhibit action thereof; variant protein, peptide and analog thereof that act as dominant negatives; siRNA, shRNA and microRNA that suppress expression of a protein of a pathway and the like.

As one of the embodiments of the TGF-β/SMAD pathway inhibitor, an inhibitor (ALK inhibitor) of ALK proteins (ALK1, ALK2, ALK3, ALK4, ALK5, ALK6, ALK7) belonging to the TGF-β type I receptor family (also referred to as Activin receptor like kinase (ALK) family) is recited. Also, an inhibitor of a protein belonging to the TGF-β type II receptor family (TGF-βII(AAT3), ACTRII, ACTRIIB, BMPRII, AMHRII) is recited.

Specific examples include D4476 (4-[4-(2,3-dihydro-1,4-benzodioxin-6-yl)-5-(2-pyridlnyl-)1H-imidazol-2-yl]-benzamide), ALK5 Inhibitor II (2-(3-(6-Methylpyridin-2-yl)-1H-pyrazol-4-yl)-1,5-naphthyridine; alias RepSox), GW788388, SD-208 as inhibitors of ALK5; LY2109761, LY2157299 (Galunisertiv, 4-[5,6-dihydro-2-(6-methyl-2-pyridinyl)-4H-pyrrolo[1,2-b]pyrazol-3-yl]-6-quinolinecarboxamide), LY364947 as inhibitors of ALK5 and TGF-βRII (AAT3); SM16 (4-(5-(benzo[d][1,3]dioxol-5-yl)-4-(6-methylpyridin-2-yl)-1H-imidazol-2-yl)bicyclo[2.2.2]octane-1-carboxamide), EW-7197, SB525334 as inhibitors of ALK4 and ALK5; SB431542 (4-[4-(1,3-Benzodioxol-5-yl)-5-(pyridin-2-yl)-1H-imidazol-2-yl]benzamide), SB505124, A83-01 as inhibitors of ALK4, ALK5 and ALK7; LDN-193189, Apigenin, DMH1, ML347 as inhibitors of ALK2 and ALK3; LDN-214117 as inhibitor of ALK1 and ALK2; LDN-212854 as inhibitor of ALK1, ALK2 and ALK3; and K02288 as inhibitor of ALK1, ALK2, ALK3 and ALK6.

As the ALK inhibitor, one having at least an inhibitory activity against ALK5 (ALK5 inhibitor) is preferable in view of the high effect. One having at least an inhibitory activity against ALK4 and ALK5, or ALK5 (of ALK proteins, one having remarkably high inhibitory activity against the protein) is preferable in view of the particularly high effect.

As another embodiment of the TGF-β/SMAD pathway inhibitor, an inhibitor of SMAD protein is recited.

Among others, an inhibitor of SMAD2 and SMAD3 located at the downstream of ALK5, further SMAD4, is preferable.

The TGF-β/SMAD pathway inhibitor also encompasses derivatives of the above-mentioned compounds. For example, a derivative of D4476 can also be used instead of D4476. The derivative does not necessarily have an ALK5 inhibitory activity. For example, a derivative of D4476 represented by the following formula (I) described in WO 00/61576 can be used:

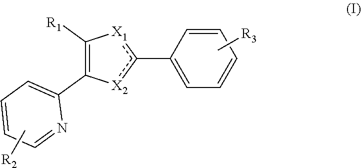

wherein R₁ is naphthyl, anthracenyl or phenyl optionally substituted by one or more substituents selected from the group consisting of a halogen, C₁₋₆ alkoxy (—O—C₁₋₆ alkyl), C₁₋₆ alkylthio (—S—C₁₋₆ alkyl), C₁₋₆ alkyl, —O—(CH₂)ₙ-Ph, —S—(CH₂)ₙ-Ph, cyano, phenyl(Ph) and CO₂R (R is hydrogen or C₁₋₆ alkyl, and n is 0, 1, 2 or 3); or R₁ is phenyl fused with a 5- to 7-membered aromatic ring or nonaromatic ring optionally containing up to two hetero atoms independently selected from N, O and S;
R₂ is H, NH(CH₂)ₙ-Ph or NH—C₁₋₆ alkyl (n is 0, 1, 2 or 3);
R₃ is CO₂H, CONH₂, CN, NO₂, C₁₋₆ alkylthio, —SO₂—C₁₋₆ alkyl, C₁₋₆ alkoxy, SONH₂, CONHOH, NH₂, CHO, CH₂OH, CH₂NH₂ or CO₂R (R is hydrogen or C₁₋₆ alkyl); one of X₁ and X₂ is N or CR′ and the other is NR′ or CHR′ (R′ is hydrogen, OH, C₁₋₆ alkyl or C₃₋₇ cycloalkyl); or when one of X₁ and X₂ is N or CR′, the other may be S or O.

Examples of C₁₋₆ alkyl include linear or branched chain alkyl having 1-6 carbon atoms, specifically, methyl, ethyl, n-propyl, isopropyl, n-butyl, isobutyl, sec-butyl, tert-butyl, n-pentyl, isopentyl, n-hexyl and isohexyl.

Examples of C₃₋₇ cycloalkyl include cyclopropyl having 3-7 carbon atoms, specifically, cyclopropyl, cyclobutyl, cyclopentyl, cyclohexyl and cycloheptyl.

When R₁ is phenyl fused with a 5- to 7-membered aromatic ring or nonaromatic ring optionally containing up to two hetero atoms independently selected from N, O and S, specific examples include benzo[1,3]dioxolyl, 2,3-dihydrobenzo[1,4]dioxynyl, benzoxazolyl, benzothiazolyl, benzo[1,2,5]oxadiazolyl, benzo[1,2,5]thiadiazolyl and dihydrobenzofuranyl.

As such derivatives of D4476, the following compounds are recited as examples:
4-[4-(4-fluorophenyl)-5-(2-pyridyl)-1-hydroxy-1H-imidazol-2-yl]benzonitrile;
4-[4-(4-fluorophenyl)-5-(2-pyridyl)-1H-imidazol-2-yl]benzonitrile;
4-[4-(4-fluorophenyl)-5-(2-pyridyl)-1H-imidazol-2-yl]benzoic acid;
methyl 4-[4-(4-fluorophenyl)-5-(2-pyridyl)-1H-imidazol-2-yl]benzoate;
ethyl 4-[4-(4-fluorophenyl)-5-(2-pyridyl)-1H-imidazol-2-yl]benzoate;
4-(4-benzo[1,3]dioxol-5-yl-1-hydroxy-5-pyridin-2-yl-1H-imidazol-2-yl)benzonitrile;
4-(4-benzo[[1,3]dioxol-5-yl-5-pyridin-2-yl-1H-imidazol-2-yl)benzonitrile;
4-(4-benzo[1,3]dioxol-5-yl-5-pyridin-2-yl-1H-imidazol-2-yl)benzoic acid;
2-[4-benzo(1,3]dioxol-5-yl-2-(4-nitrophenyl)-1H-imidazol-5-yl]pyridine;
3-(4-benzo[1,3]dioxol-5-yl-5-pyridin-2-yl-1H-imidazol-2-yl)phenylamine;
4-[4-(4-fluorophenyl)-2-(4-nitrophenyl)-1H-imidazol-5-yl]pyridine;
4-[4-(4-fluorophenyl)-5-pyridin-2-yl-1H-imidazol-2-yl)phenylamine;
4-(4-benzo[1,3]dioxol-5-yl-5-pyridin-2-yl-1H-imidazol-2-yl)phenyl]methanol;
4-(4-benzo[1,3]dioxol-5-yl-5-pyridin-2-yl-1H-imidazol-2-yl)benzamide;
4-[4-(2,3-dihydro-benzo[1,4]dioxin-6-yl)-5-pyridin-2-yl-1H-imidazol-2-yl]-benzonitrile;
4-[4-(2,3-dihydro-benzofuran-5-yl)-5-pyridin-2-yl-1H-imidazol-2-yl]benzamide;
3-[4-benzo[1,3]dioxol-5-yl-5-pyridin-2-yl-1H-imidazol-2-yl)benzonitrile;
4-[4-(2,3-dihydro-benzofuran-6-yl)-5-pyridin-2-yl-1H-imidazol-2-yl]benzonitrile;
4-[4-(2,3-dihydro-benzofuran-6-yl)-5-pyridin-2-yl-1H-imidazol-2-yl]benzamide;
3-(4-benzo[1,3]dioxol-5-yl-5-pyridin-2-yl-1H-imidazol-2-yl)benzoic acid;
4-(4-(4-methoxyphenyl)-5-(2-pyridyl)-1H-imidazol-2-yl]benzonitrile;
4-[4-(2,2-difluoro-benzo[1,3]dioxol-5-yl)-5-pyridin-2-yl-1H-imidazol-2-yl]benzamide;
4-[4-(2,3-dihydro-benzo[1,4]dioxin-6-yl)-1-methyl-5-pyridin-2-yl-1H-imidazol-2-yl]benzamide;
4-[5-(2,3-dihydro-benzo[1,4]dioxin-6-yl)-1-methyl-4-pyridin-2-yl-1H-imidazol-2-yl]benzamide;
4-(5-benzo[1,3]dioxol-5-yl-4-pyridin-2-yl-oxazol-2-yl)benzonitrile;
4-(5-benzo[1,3]dioxol-5-yl-4-pyridin-2-yl-oxazol-2-yl)benzamide; and 4-(4-benzo[1,3]dioxol-5-yl-5-pyridin-2-yl-1H-pyrrol-2-yl)benzamide.

### Casein Kinase Inhibitor

Casein kinase inhibitor widely encompasses inhibitors against casein kinase having subtypes such as casein kinase 1, casein kinase 2 and the like. Casein kinase inhibitor encompasses not only low-molecular-weight compounds which are inhibitors in the narrow sense but also antibody, aptamer and peptide that bind to a casein kinase and have an activity to inhibit action thereof; variant protein and analog thereof that act as dominant negatives; siRNA, shRNA and microRNA that suppress expression of a casein kinase and the like.

Casein kinase 1 inhibitor is a preferable embodiment in view of the high brown adipocyte induction effect.

Preferable examples of the casein kinase 1 inhibitor include compounds such as D4476, IC261, CK1-7, A3, SB-431542, DRB, hymenialdisine, matairesinol, 5-iodotubercidin, meridian, SB-203580 and the like (including compounds specifically inhibiting casein kinase 1).

Besides these, compounds having an activity to inhibit casein kinase 1 such as fasudil, hydroxyfasudil, fenretinide, PKZ-ζ peptide pseudosubstrate, dimethylsphingosine, CVS-3989, AG1024, 648450, K252a, C3 transferase, 553502, LY333531, ruboxistaurin, Go-6976, IWR-1-endo (IWR1e), IWP-2 and the like can also be mentioned.

As the casein kinase 2 inhibitor, CX-4945 can be mentioned.

Casein kinase inhibitor also encompasses derivatives of the above-mentioned compounds.

cAMP Inducer

A cAMP inducer (which can also be referred to as an adenylate cyclase activator) widely encompasses a compound that increases the level of intracellular cAMP (cyclic AMP) by the activation action of adenylate cyclase. Examples thereof include forskolin (FSK), isoproterenol, NKH 477, PACAP 1-27, PACAP 1-38 and the like.

The cAMP inducer also encompasses derivatives of the above-mentioned compounds.

### MEK/ERK Pathway Inhibitor

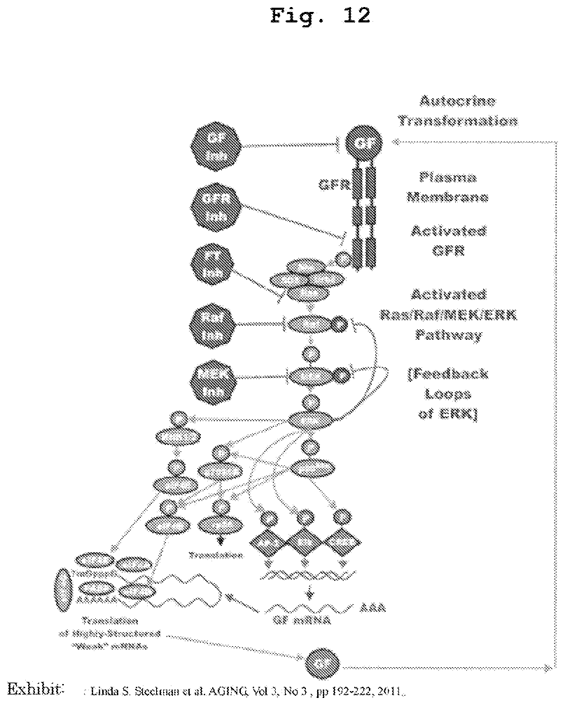

MEK/ERK pathway inhibitor means a compound capable of inhibiting the functional expression of protein belonging to the MEK/ERK pathway. The MEK/ERK pathway is a signal pathway known to those of ordinary skill in the art and is shown in FIG. 12.

MEK/ERK pathway is mainly constituted of receptors such as EGF receptor, HER2, IGF1 receptor, VEGF receptor, Flt-3, c-kit, PDGF-R and the like, which are activated by the binding of cytokine and growth factors; Ras activated by these receptors; A-Raf, B-Raf, c-Raf, Mos, Tp1 which are MAPKKK proteins activated by Ras signal; MEK1, MEK2 (MEK1/2) which are MAPKK proteins phosphorylated (activated) by MAPKKK, ERK1, ERK2 (ERK1/2) which are MAPK proteins phosphorylated (activated) by MAPKK; Elk-1, Est2, RSK, MNK, MSK, cPLA2, CREB, Fos, globin transcription factor 1 which are transcription factors at the downstream, and the like.

The MEK/ERK pathway inhibitor includes one that inhibits any of the above-mentioned molecules (cytokine, growth factor and receptor thereof at the upstream of MEK, Ras, Raf, MEK1/2, ERK1/2, a factor at the downstream of ERK etc.). Among others, a compound (inhibitor) that inhibits functional expression of MEK1, MEK2 of the MAPKK protein and, ERK1, ERK2 of the MAPK protein is preferable, and an inhibitor of MEK1, MEK2 is particularly preferable.

Examples of the MEK/ERK pathway inhibitor include PD0325901 (N-[(2R)-2,3-dihydroxypropoxy-3,4-difluoro-2-[(2-fluoro-4-iodophenyl)amino]-benzamide; inhibitor of MEK1/2), AS703026, AZD8330, BIX02188, BIX02189, CI-1040, Cobimetinib, GDC-0623, MEK162, PD318088, PD98059, Refametinib, RO4987655, SCH772984, Selumetinib, SL327, Trametinib, ARRY-142886, XL518, RDEA119 and the like.

In addition, the MEK/ERK pathway inhibitor also encompasses derivatives of the above-mentioned compounds. The MEK/ERK pathway inhibitor encompasses not only low-molecular-weight compounds which are inhibitors in the narrow sense but also antibody, aptamer and peptide that bind to a protein of MEK/ERK pathway (e.g., MEK1, MEK2, ERK1, ERK2) and have an activity to inhibit action thereof; variant protein and analog thereof that act as dominant negatives; siRNA, shRNA and microRNA that suppress expression of a protein of MEK/ERK pathway (e.g., MEK1, MEK2, ERK1, ERK2) and the like.

The concentration of the compound selected from the group consisting of the above-mentioned (1)-(4) in a medium can be appropriately determined by those of ordinary skill in the art. It is generally about 0.01 μM-100 μM, particularly about 0.1 μM-10 μM.

### Culture

In the method of the present invention, a differentiated somatic cell of a mammal is cultured in a medium in the presence of at least one kind of a compound selected from the group consisting of the above-mentioned (1)-(4).

Cultivation can be performed in an appropriate container for storing cells and media. A method for performing preferable culture is, for example, a culture method under conditions of about 37° C. and carbon dioxide concentration of about 5%, though the method is not limited thereto. Culture under the above-mentioned conditions can be performed using, for example, a known CO₂ incubator.

S At least one kind of compound selected from the group consisting of the above-mentioned (1)-(4) may be added only in a part of the period in the whole culture period. Differentiated somatic cells of a mammal may be cultured in the presence of the above-mentioned compound in a normal medium and then cultured in the absence of the above-mentioned compound in an induction medium. Alternatively, after culturing in the presence of the above-mentioned compound in a normal medium, the cells may be cultured in the absence of the above-mentioned compound in a normal medium and then cultured in the absence of the above-mentioned compound in the induction medium. Alternatively, after culturing in the presence of the above-mentioned compound in a normal medium, the cells may be cultured in the presence of the above-mentioned compound in an induction medium and then cultured in the absence of the above-mentioned compound in the induction medium. Thus, as long as both processes of culturing in the presence of the above-mentioned compound and culturing in an induction medium are included, they may not be performed simultaneously and each may be performed only in a part of the whole culture period.

The period of culturing is not particularly limited as long as the effect of the present invention is not impaired. For example, it can be set to 24 hr to about 60 days, preferably 3-30 days, more preferably about 10-20 days, particularly preferably about 14 days.

In view of the high effect, in the whole culture period, it is possible to adopt culturing in the presence of the above-mentioned compound in an induction medium (e.g., about 6-10 days, particularly about 8 days) and then culturing in the absence of the above-mentioned compound in an induction medium. In this case, in the whole culture period, culturing in the presence of the above-mentioned compound may be from the start of culturing or after culturing in the absence of the above-mentioned compound for a given period.

In culturing, passage can be performed as necessary. When passage is performed, cells are recovered before or immediately after reaching the confluence and seeded in a fresh medium. The medium can also be changed as appropriate in culturing in the present invention.

In this way, somatic cell is converted to brown adipocyte and brown adipocyte is generated.

Obtainment of the brown adipocyte can be confirmed by the aforementioned staining with fluorescent dye capable of detecting lipid droplets in cells or detection of gene products expressed in brown adipocytes.

To be specific, obtainment of brown adipocyte can be detected by possible staining by Oil Red O staining or Bodipy staining, unique shape with multilocular lipid droplets, expression of UCP-1, CIDEA, KCNK3, PCG-1α, Cox8b, Otop, ELOVL3 gene and the like. Among others, UCP-1 (Uncoupling protein 1) is a gene specifically expressed in brown adipocytes, encodes mitochondrial inner membrane protein that uncouples oxidative phosphorylation, and is considered to be the basis of the function of brown adipocytes. Thus, it is one of the particularly preferable ones as indices of brown adipocytes.

### Treatment or Prophylactic Agent; Transplantation Material

The brown adipocyte generated by the method of the present invention can be used for the prophylaxis or treatment of obesity, metabolic syndrome or disease or condition related to these, by transplantation to the body.

The target disease includes Type I diabetes, Type II diabetes, diabetic complications (retinopathy, peripheral neurosis, nephropathy, macroangiopathy, diabetic gangrene, osteoporosis, diabetic coma etc.), impaired glucose tolerance, insulin resistance, acidosis, ketosis, ketoacidosis, obesity, central obesity and complications thereof, visceral obesity syndrome, hypertension, postprandial hyperlipidemia, cerebrovascular diseases, arteriosclerosis, atherosclerosis, metabolic-syndrome, dyslipidemia, hypertriglyceridemia, hypercholesterolemia, hypoHDL-emia, renal, disease (diabetic nephropathy, nephrotic syndrome etc.), arteriosclerosis, thrombotic disease, myocardial infarction, ischemic cardiac diseases, angina pectoris, cardiac failure, cerebrovascular diseases (cerebral infarction, cerebral apoplexy etc.), peripheral blood circulation disorder, perception disorder, hyperuricemia, gout, infections (respiratory infection, urinary tract infection, gastrointestinal infections, skin infections, soft tissue infections etc.), malignant tumor, cataract, fatty liver, non-alcoholic steatohepatitis and osteoporosis. Prophylactic or treatment effects on these diseases are considered to be obtained due to lipid burning and improvement of sugar and lipid metabolism abnormality by brown adipocytes.

In addition, brown adipocytes can also be used for cosmetic application to remove fat around the abdomen and jaw, of the thigh and the like. Brown adipocytes can also be used as a transplantation material to be introduced into breast and the like for cosmetic applications.

When brown adipocytes are administered, fat content, particularly white adipocytes such as visceral fat, subcutaneous fat and the like decrease, and the body weight increase is suppressed when a high-calorie food is ingested. Therefore, brown adipocytes are useful for both the prophylaxis and treatment of obesity, metabolic syndrome or disease or condition related to these. The present invention can also be used not only for the prophylaxis or treatment of diseases but also health promotion and beauty (e.g., removal of visceral fat and subcutaneous fat in abdomen, jaw, arm, thigh and the like) and the like. In this case, dealing of human is conveniently referred to as treatment in the present specification, and “patient” can mean “healthy human” or “human” and “disease” can mean “health promotion”, “beauty” and the like.

The present invention can also be used for the treatment of diseases in not only human but also pet animals such as dog, cat and the like and domestic animals such as bovine, horse, swine, sheep, chicken and the like. In this case, “patients” and “human” are respectively referred to as “animal patient” and “animal”.

The transplantation material refers to a material that introduces brown adipocytes into the body. Brown adipocyte can also be used as a transplantation material to be introduced into breast and the like for cosmetic applications. The transplantation material encompasses a material to be transplanted to the same or different individual after conversion of somatic cell to brown adipocyte in vitro.

Using the obtained brown adipocytes, the drug discovery and development and the like based on a new action mechanism for diabetes (particularly type II diabetes), impaired glucose tolerance, lipid metabolism abnormality, arteriosclerotic disease, hypertension, hyperuricemia, gout, non-alcoholic steatohepatitis and the like can be performed.

### EXAMPLES

While the Examples are shown below, the present invention is not limited to the Examples alone.

The structures of the compounds used in the Examples are shown below.

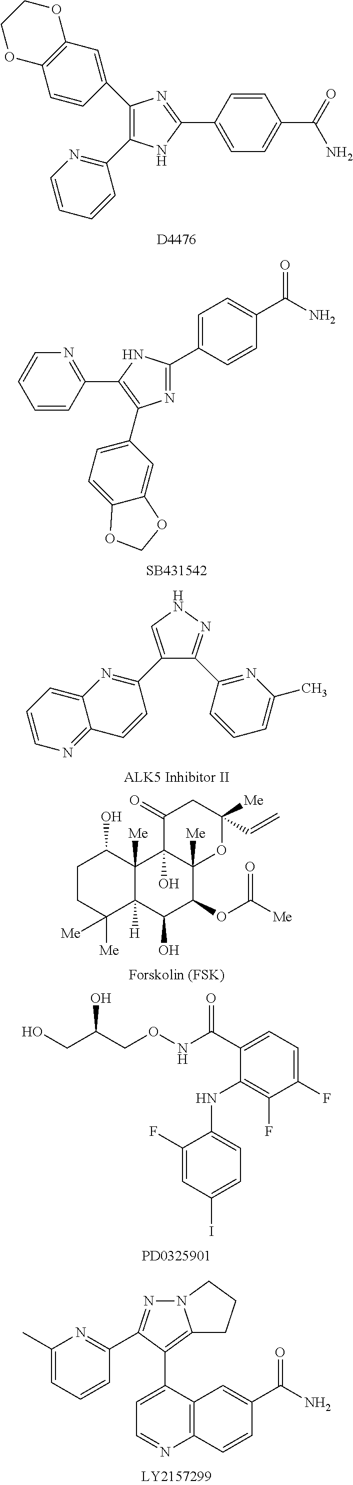

In the present specification and drawings below, “ALK5 Inhibitor II” is sometimes indicated as “ALK5 Inhibitor”, “ALK5IH” or “ALK5i”.

### Example 1

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humidified air, 37° C. The next day, the culture supernatant was removed by suction and, as described in the Figure, a normal medium, an adipocyte induction medium, or an adipocyte induction medium added with the compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 μM dexamethason and 1 μg/mL Insulin).

The concentrations of the additives are as follows:
T3: 1 nM
Rosiglitazone: 1 μM
D4476: 2 μM
Pifithrin alpha [p53 inhibitor]: 5 μM
SB431542: 2 μM
ALK5 Inhibitor II: 2 M.

The culture medium was substituted by a fresh one every 3-4 days and the cells were cultured up to day 14.

On day 14, the culture medium was removed from each well by suction, the cells were washed with PBS(−) and fixed with 10% formalin. After washing 3 times with sterile distilled water, Oil Red O staining solution was added, and the mixture was incubated at room temperature for 15 min. Then, the cells were washed with sterile distilled water and photographed at a magnification of 100 with a phase contrast microscope.

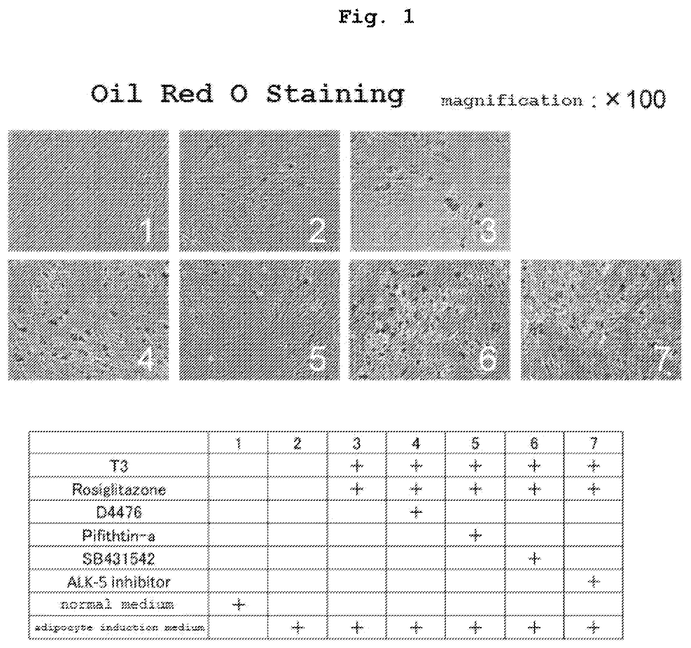

The results are shown in FIG. 1. Remarkable Oil Red O staining was observed when any of D4476, SB431542 and ALK5 Inhibitor II was added to the adipocyte induction medium in addition to T3 and Rosiglitazone and the cells were cultured (In FIG. #4, #6, #7). On the other hand, Oil Red O staining was hardly observed in the normal medium, adipocyte induction medium without addition of T3 and Rosiglitazone, and adipocyte induction medium added with Pifithrin alpha (p53 inhibitor). The level of Oil Red O staining was low in adipocyte induction medium added with T3 and Rosiglitazone alone. From the above, it is clear that fibroblasts were converted to brown adipocytes when any of D4476, SB431542 and ALK5 Inhibitor II was added in addition to T3 and Rosiglitazone and the cells were cultured.

### Example 2

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humid air, 37° C. The next day, the culture supernatant was removed by suction and, as described in the Figure, a normal medium, an adipocyte induction medium, or an adipocyte induction medium added with each low-molecular compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 μM dexamethason and 1 μg/mL Insulin).

The concentrations of the additives are as follows:
T3: 1 nM
Rosiglitazone: 1 μM
D4476: 2 μM
Pifithrin alpha [p53 inhibitor]: 5 μM
Forskolin (FSK): 2 μM
PD0325901: 1 μM
SB431542: 2 μM.

The culture medium was substituted by a fresh one every 3-4 days and the cells were cultured up to day 14.

On day 14, the culture medium was removed from each well by suction, the cells were washed with PBS(−) and total RNA was extracted from the cells with ISOGEN II. cDNA was synthesized from the RNA by using Rever Tra Ace qPCR RT Master Mix. The cDNA was admixed with Real-time PCR Master Mix, primers specific to UCP1 gene or β actin gene and Tagman probe. qRT-PCR (quantitative RT-PCR) was performed using AB7300 Real-time PCR system. The mRNA level of UCP1 gene was quantified as a ratio to β actin gene mRNA and calculated with the value of fibroblast cultured in the normal medium as 1.

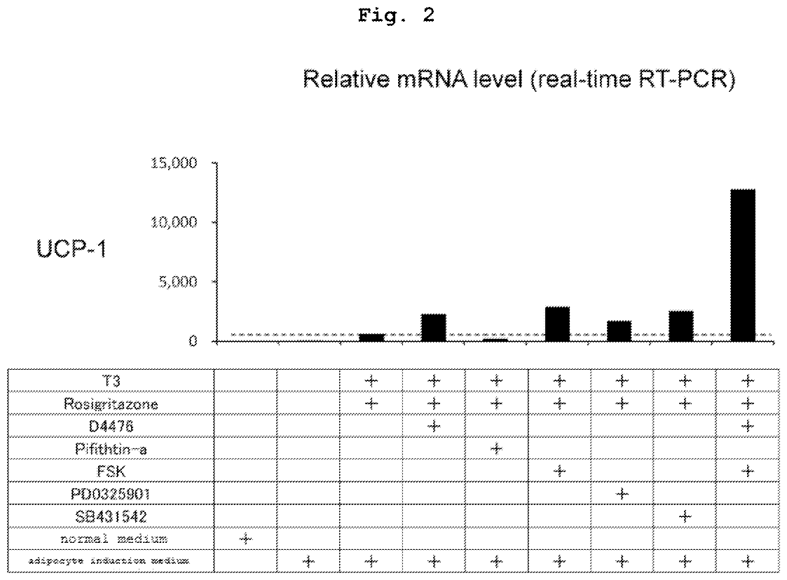

The results thereof are shown in FIG. 2. It is clear that fibroblasts were induced to brown adipocytes that express mRNA of UCP1 gene when any of D4476, FSK, PD0325901 and SB431542 was added in addition to T3 and Rosiglitazone and the cells were cultured. Furthermore, it is clear that coaddition of D4476 and FSK caused conversion to a cell that expresses UCP1 most strongly.

### Example 3

An experiment similar to that in Example 2 was performed, and cells cultured in a normal medium, cells cultured for 14 days in an adipocyte induction medium added with T3 and Rosiglitazone, and cells cultured for 14 days in an adipocyte induction medium added with T3, Rosiglitazone and D4476 were prepared. 10 μM Isoproterenol or FSK was added to these cells as described in the Figure. As a control, a group free of the addition was also prepared. After 5 hr, the culture medium was removed from each well by suction, the cells were washed with PBS(−) and total RNA was extracted from the cells with ISOGEN II. qRT-PCR was performed in the same manner as in Example 2. The mRNA level of UCP1 gene was quantified as a ratio to @actin gene mRNA and calculated with the value of fibroblast cultured in the normal medium as 1.

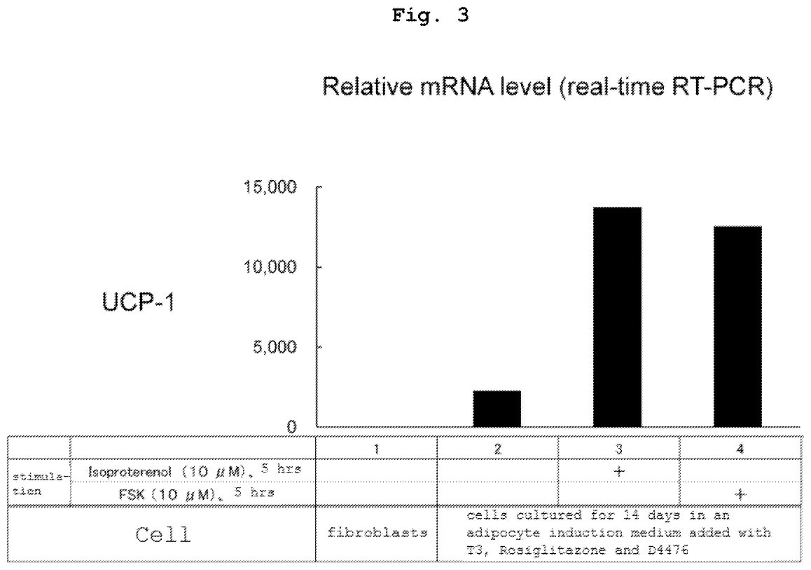

The results thereof are shown in FIG. 3. It is clear that the cells cultured for 14 days in an adipocyte induction medium added with D4476 in addition to T3 and Rosiglitazone more strongly express UCP1 mRNA by stimulation with Isoproterenol or FSK, and have brown adipocyte-like responsiveness to these stimuli.

### Example 4

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humidified air, 37° C. The next day, the culture supernatant was removed by suction and, as described in the Figure, a normal medium, an adipocyte induction medium, or an adipocyte induction medium added with each compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 μM dexamethason and 1 μg/mL Insulin).

The concentrations of the additives are as follows:
T3: 1 nM
Rosiglitazone: 1 μM
D4476: 2 μM
Pifithrin alpha [p53 inhibitor]: 5 μM
SB431542: 2 μM
ALK5 Inhibitor II: 2 μM.

The culture medium was substituted by a fresh one every 3 4 days and the cells were cultured up to day 14. On day 14, the culture medium was removed from each well by suction, the cells were washed with PBS(−) and total RNA was extracted from the cells with ISOGEN II. cDNA was synthesized from the RNA by using Rever Tra Ace qPCR RT Master Mix. The cDNA was admixed with Real-time PCR Master Mix, primers specific to CIDEA gene or β actin gene and Tagman probe. qRT-PCR was performed using AB7300 Real-time PCR system. The mRNA level of CIDEA gene was quantified as a ratio to R actin gene mRNA and calculated with the value of fibroblast cultured in the normal medium as 1.

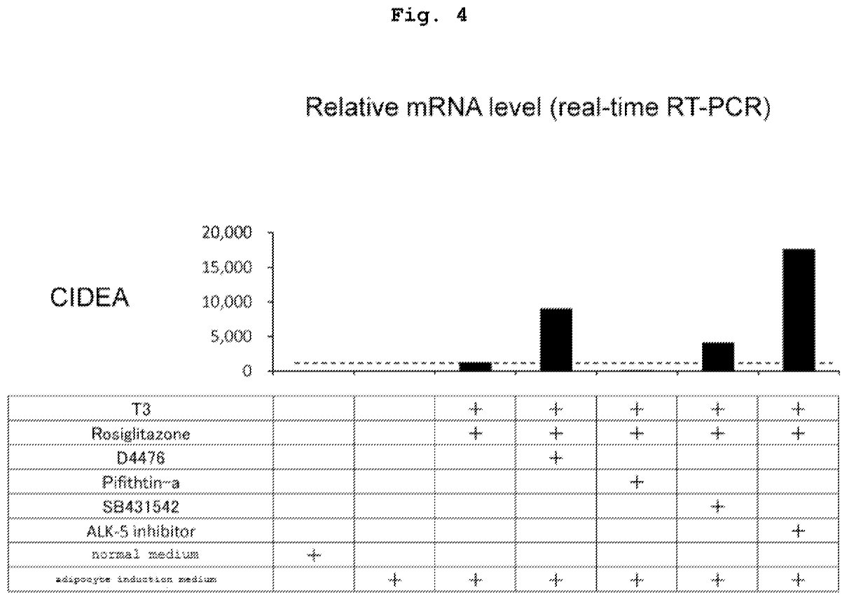

The results thereof are shown in FIG. 4. It is clear that fibroblasts were converted to brown adipocytes expressing mRNA of CIDEA gene by the addition culture with any of D4476, SB431542 and ALK5 Inhibitor in addition to T3 and Rosiglitazone.

### Example 5

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humidified air, 37° C. The next day, the culture supernatant was removed by suction and, as described in the Figure, a normal medium, an adipocyte induction medium, or an adipocyte induction medium added with each compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 μM dexamethason and 1 μg/mL Insulin).

The concentrations of the additives are as follows:
T3: 1 nM
Rosiglitazone: 1 μM
Pifithrin alpha [p53 inhibitor]: 5 μM
Forskolin (FSK): 2 μM
PD0325901: 1 μM.

The culture medium was substituted by a fresh one every 3-4 days and the cells were cultured up to day 14.

On day 14, the culture medium was removed from each well by suction, the cells were washed with PBS(−) and total RNA was extracted from the cells with ISOGEN II. cDNA was synthesized from the RNA by using Rever Tra Ace qPCR RT Master Mix. The cDNA was admixed with Real-time PCR Master Mix, primers specific to PGC-1alpha or β actin gene and Taqman probe. qRT-PCR was performed using AB7300 Real-time PCR system. The mRNA level of PGC-1alpha gene was quantified as a ratio to β actin gene mRNA and calculated with the value of fibroblast cultured in the normal medium as 1.

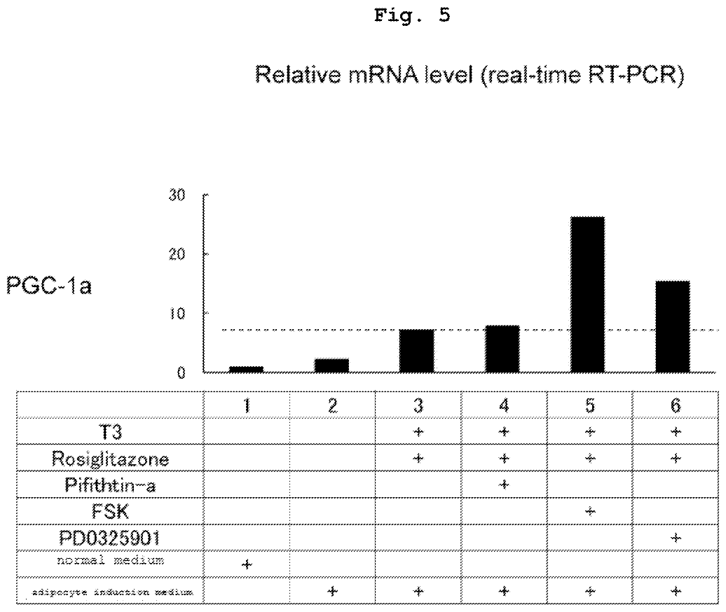

The results thereof are shown in FIG. 5. It is clear that fibroblasts were converted to brown adipocytes expressing mRNA of PGC-1alpha gene by the addition culture with any of Forskolin (FSK) and PD0325901 in addition to T3 and Rosiglitazone.

### Example 6

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humid air, 37° C. The next day, the culture supernatant was removed by suction and, as described in the Figure, a normal medium, an adipocyte induction medium, or an adipocyte induction medium added with each low-molecular-weight compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 μM dexamethason and 1 μg/mL Insulin).

The concentrations of the additives are as follows:
T3: 1 nM
Rosiglitazone: 1 μM
D4476: 2 μM.
Pifithrin alpha [p53 inhibitor]: 5 μM
PD0325901: 1 μM
SB431542: 2 μM
ALK5 Inhibitor II: 2 μM.

The culture medium was substituted by a fresh one every 3-4 days and the cells were cultured up to day 14.

On day 14, the culture medium was removed from each well by suction, the cells were washed with PBS(−) and total RNA was extracted from the cells with ISOGEN II. cDNA was synthesized from the RNA by using Rever Tra Ace qPCR RT Master Mix. The cDNA was admixed with Real-time PCR Master Mix, primers specific to AdipoQ or a actin gene and Taqman probe. qRT-PCR was performed using AB7300 Real-time PCR system. The mRNA level of AdipoQ gene was quantified as a ratio to Q actin gene mRNA and calculated with the value of fibroblast cultured in the normal medium as 1.

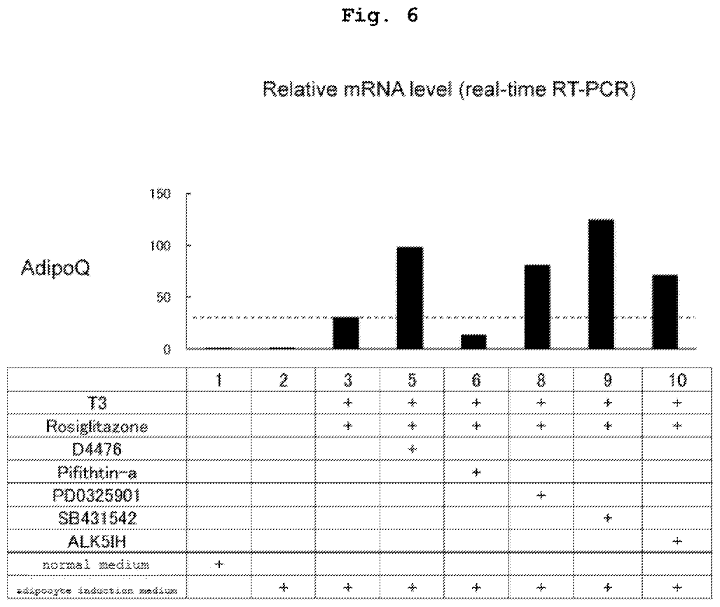

The results thereof are shown in FIG. 6. It is clear that fibroblasts were converted to brown adipocytes expressing mRNA of AdipoQ gene by the addition culture with any of D4476, PD0325901, SB431542 and ALK5 Inhibitor II in addition to T3 and Rosiglitazone.

### Example 7

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humidified air, 37° C. The next day, the culture supernatant was removed by suction and, as described in the Figure, a normal medium, an adipocyte induction medium, or an adipocyte induction medium added with each low-molecular-weight compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 uM dexamethason and 1 μg/mL Insulin).

The concentrations of the additives are as follows:
T3: 1 nM
Rosiglitazone: 1 μM
D4476: 2 μM
SB431541: 2 μM
ALK5 inhibitor II: 2 μM.

The culture medium was substituted by a fresh one every 3-4 days and the cells were cultured up to day 14.

On day 14, the culture medium was removed from each well by suction, and the cells were washed with PBS(−). The cells were fixed with 4% para-formaldehyde, washed with PBS(−), reacted for 5 min with BODIPY 493/503 (Invitrogen)/PBS solution at room temperature and washed 3 times with PBS. The cells were photographed at a magnification of 200 with a fluorescence microscope and the fluorescence intensity was measured.

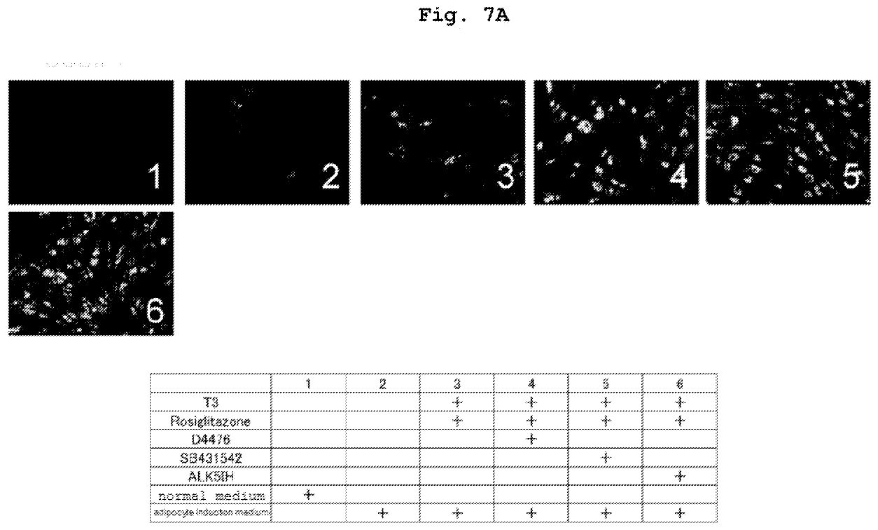

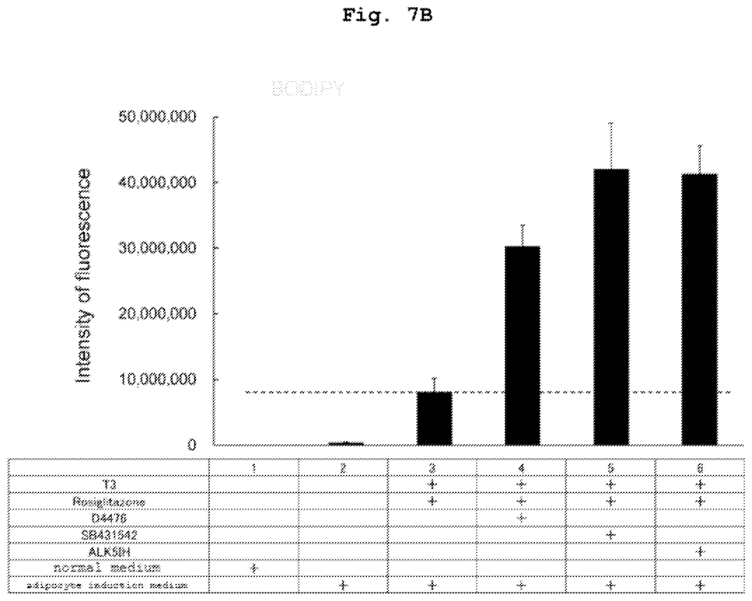

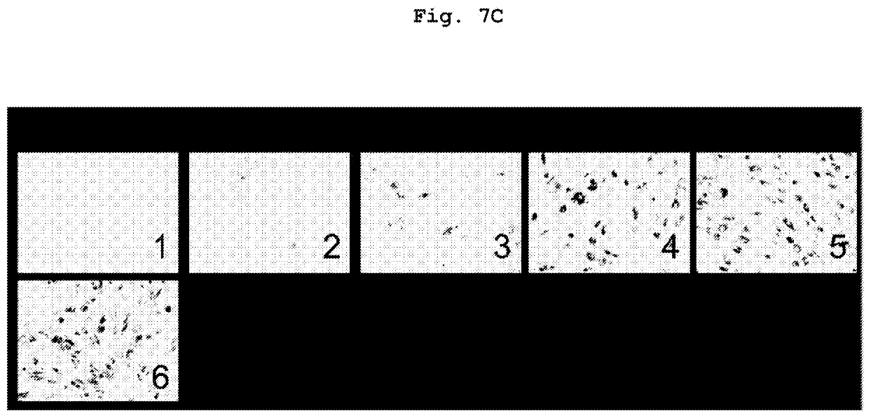

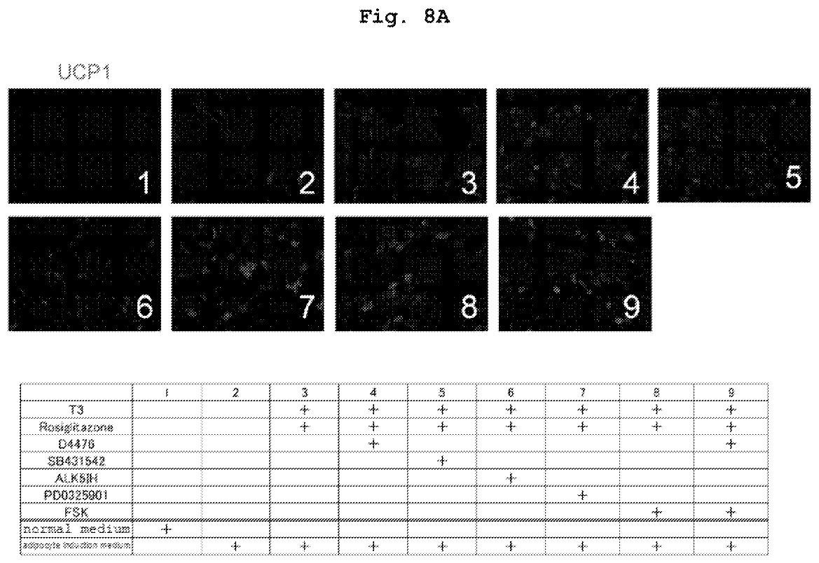

The results thereof are shown in FIG. 7A (fluorescence microscopic images) and FIG. 7B (fluorescence intensity). It is clear that fibroblasts were converted to brown adipocytes having lipid droplets stained with BODIPY by the addition culture with any of D4476, SB431541 and ALK5 Inhibitor II in addition to T3 and Rosiglitazone.

### Example 8

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humidified air, 37° C. The next day, the culture supernatant was removed by suction and, as described in the Figure, a normal medium, an adipocyte induction medium, or an adipocyte induction medium added with each compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 μM dexamethason and 1 μg/mL Insulin).

The concentrations of the additives are as follows:
T3: 1 nM
Rosiglitazone: 1 μM
D4476: 2 μM
SB431541: 2 μM
ALK5 inhibitor II: 2 μM
PD0325901: 1 μM
Forskolin (FSK): 2 μM.

The culture medium was substituted by a fresh one every 3-4 days and the cells were cultured up to day 14.

On day 14, the culture medium was removed from each well by suction, and the cells were washed with PBS(−). The cells were fixed with 4% para-formaldehyde, washed with PBS(−), Perm Buffer (0.2% Triton-X-added PBS) was added and the cells were incubated for 15 min. After washing 3 times with PBS(−), Blocking One was added and the cells were incubated at room temperature for 60 min.

An anti-USP-1 antibody was added and the mixture was reacted at room temperature for 2 hr and washed 3 times with Wash buffer. Alexa 546-conjugated anti-mouse Ig antibody was added and the mixture was reacted at room temperature for 1 hr and washed 5 times with Wash buffer. The cells were photographed at a magnification of 200 with a fluorescence microscope and the fluorescence intensity was measured.

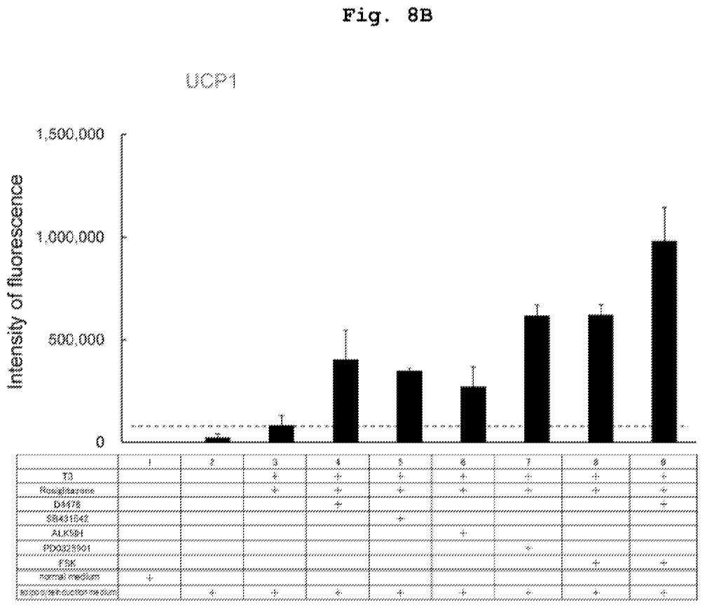

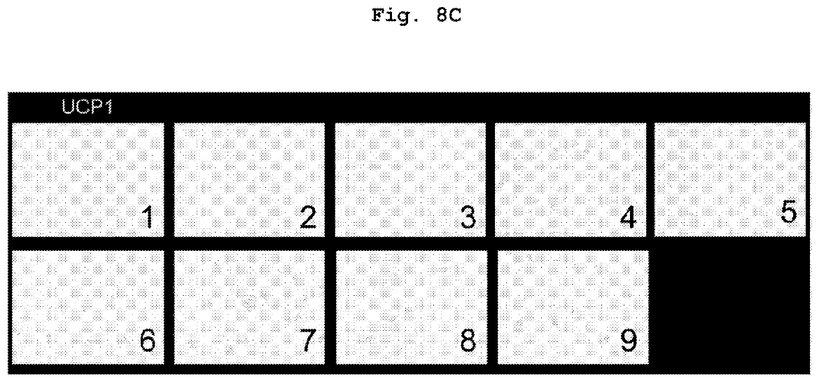

The results thereof are shown in FIG. 8A and FIG. 8C (fluorescence microscopic images) and FIG. 8B (fluorescence intensity). It is clear that fibroblasts were converted to brown adipocytes expressing UCP1 protein by the addition culture with any of D4476, SB431541, ALK5 inhibitor II, PD0325901 and Forskolin (FSK) in addition to T3 and Rosiglitazone. In addition, it is clear that expression of UCP-1 protein increases by the addition culture with PD0325901 or Forskolin. Furthermore, it is clear that fibroblasts were converted to brown adipocytes expressing UCP1 protein more strongly by the coaddition culture of D4476 and Forskolin in addition to T3 and Rosiglitazone.

### Example 9

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humidified air, 37° C. The next day, the culture supernatant was removed by suction and, as described in the Figure, a normal medium, an adipocyte induction medium, or an adipocyte induction medium added with each low-molecular-weight compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 μM dexamethason and 1 μg/mL Insulin).

The concentrations of the additives are as follows:
T3: 1 nM
Rosiglitazone: 1 μM
D4476: 2 μM.

The culture medium was substituted by a fresh one every 3-4 days and the cells were cultured up to day 14.

On day 14, the culture medium was removed from each well by suction, and the cells were washed with PBS(−) and fixed with 10% formalin. The cells were washed 3 times with sterile distilled water, Oil Red O staining solution was added, and the mixture was incubated at room temperature for 15 min. Then the cells were washed with sterile distilled water and photographed at a magnification of 100 with a microscope.

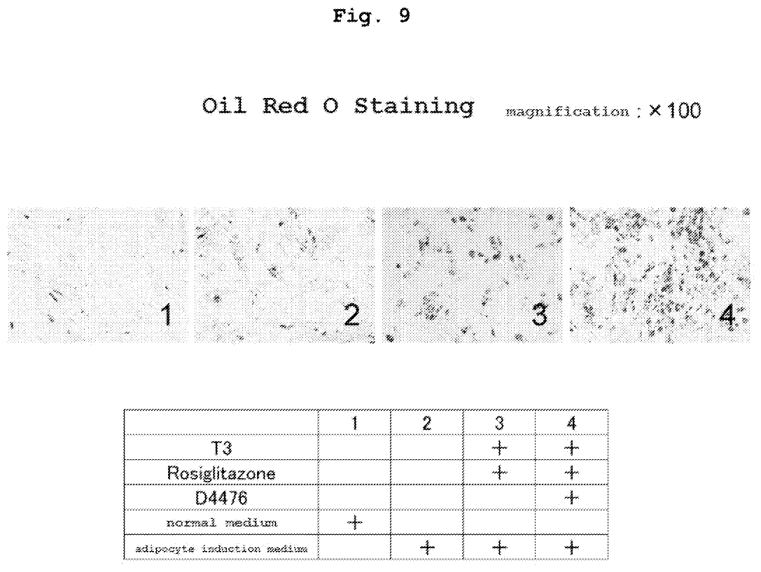

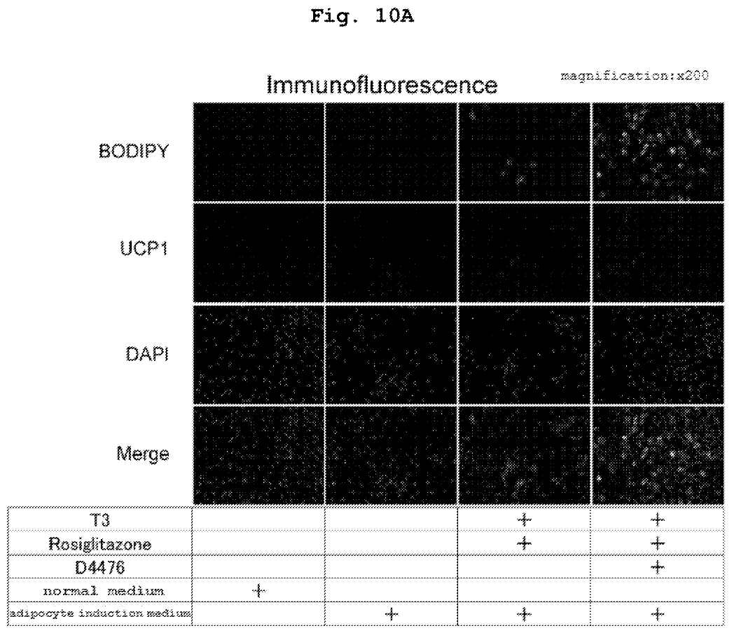

The results are shown in FIG. 9. It is clear that fibroblasts were converted to brown adipocytes showing remarkable Oil Red O staining as compared to the control by the addition culture with D4476 in addition to Rosiglitazone and T3.

### Example 10

Human normal skin-derived fibroblast (human dermal fibroblasts; HDFs) were suspended in a normal medium (10% FBS-added Dulbecco's modified minimum essential medium; DMEM). This was seeded in a 24-well plate at a concentration of 1×10⁴ cells/well (day 0), and culturing was started at 5% CO₂/95% humidified air, 37° C. The next day, the culture supernatant was removed by suction and, as described in the Figure, a normal medium, an adipocyte induction medium, or an adipocyte induction medium added with each low-molecular-weight compound and the like was added at 500 μL/well.

The adipocyte induction medium is a 10% FBS-added DMEM+MDI medium (10% FBS-added DMEM supplemented with 0.5 mM isobutylmethylxanthine (IBMX), 0.5 μM dexamethason and 1 μg/mL Insulin).

The concentrations of the additives are as follows:
T3: 1 nM
Rosiglitazone: 1 μM
D4476: 2 μM.

The culture medium was substituted by a fresh one every 3-4 days and the cells were cultured up to day 14.

On day 14, the culture medium was removed from each well by suction, and the cells were washed with PBS(−). The cells were fixed with 4% para-formaldehyde and washed with PBS(−). Perm Buffer (0.2% Triton-X-added PBS) was added and the cells were incubated for 15 min. The cells were washed 3 times with PBS(−), Blocking One was added and the cells were incubated at room temperature for 60 min.

An anti-USP-1 antibody was added and the mixture was reacted at room temperature for 2 hr and washed 3 times with Wash buffer. Alexa 546-conjugated anti-mouse Ig antibody was added and the mixture was reacted at room temperature for 1 hr and washed 5 times with Wash buffer. Then, the cells were reacted in BODIPY 493/503 (Invitrogen)/PBS solution at room temperature for 5 min, washed 3 times with PBS and stained with DAPI. The cells were photographed at a magnification of 200 with a fluorescence microscope.

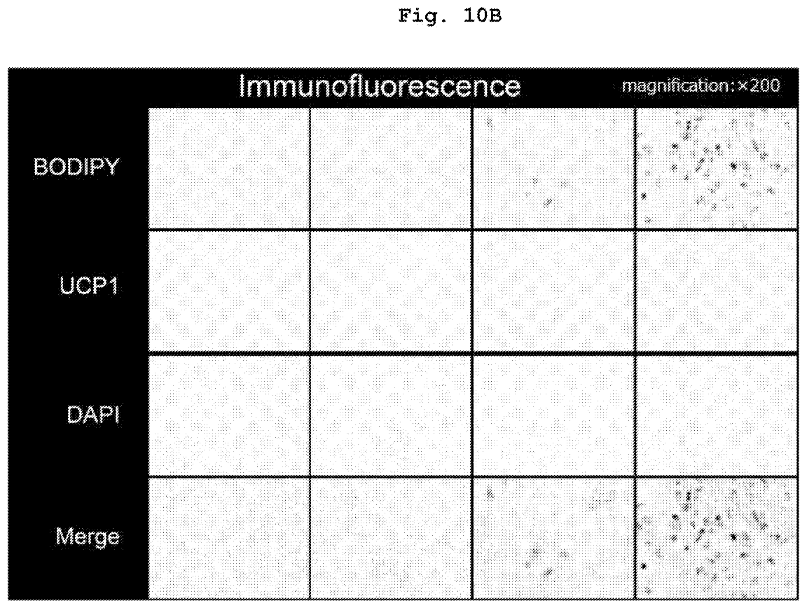

The results thereof are shown in FIG. 10A and FIG. 10B. It is clear that fibroblasts were converted to brown adipocytes expressing lipid droplets stained with Bodipy and UCP1 protein by the addition culture with D4476 in addition to T3 and Rosiglitazone.

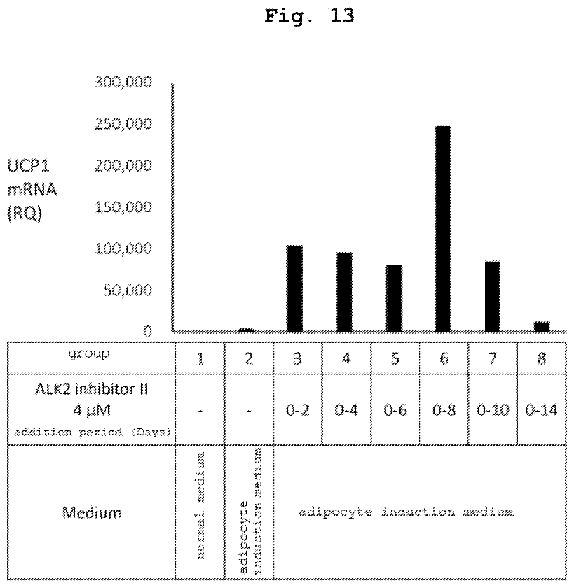

### Example 13 (FIG. 13)

Human normal skin-derived fibroblast (human dermal fibroblasts; HDFs) were suspended in a normal medium (10% FBS-added Dulbecco's modified minimum essential medium; DMEM). This was seeded in a 12-well plate at a concentration of 3×10⁴ cells/well and culturing was started at 5% CO₂/95% humidified air, 37° C. The next day (day 0), the culture supernatant was removed by suction and a normal medium (group 1), an adipocyte induction medium (group 2), or an adipocyte induction medium (groups 3-8) added with ALK5 inhibitor II at concentration of 4 μM was added at 1 mL/well.

The adipocyte induction medium is DMEM added with 1 nM T3, 1 μM Rosiglitazone, 0.5 mM isobutylmethylxanthine (IBMX), 0.5 μM dexamethason, 1 μg/mL Insulin and 10% FBS.

Once every 2 days, the medium was substituted by a fresh one. In groups 3-7, the cells were cultured in an adipocyte induction medium added with ALK5 inhibitor II only in the periods of Days 0-2, Days 0-4, Days 0-6, Days 0-8 and Days 0-10, respectively, and thereafter cultured in an adipocyte induction medium without addition of ALK5 inhibitor II. In group 8, the cells were cultured in an adipocyte induction medium added with ALK5 inhibitor II throughout the whole period of Days 0-14. On day 14, the medium was removed from each well by suction, the cells were washed with PBS(−) and total RNA was extracted from the cells by using RNA easy Mini Kit manufactured by Qiagen. cDNA was synthesized from the RNA by using Rever Tra Ace qPCR RT Master Mix. The cDNA was admixed with Real-time PCR Master Mix, primers specific to UCP1 gene or β actin gene and Taqman probe. qRT-PCR was performed using AB7300 Real-time PCR system. The mRNA level of UCP1 gene was quantified as a ratio to β actin gene mRNA and calculated with the value of fibroblast cultured in the normal medium as 1.

The results are shown in FIG. 13. It is clear that fibroblasts were converted to brown adipocytes strongly expressing UCP1 gene by the addition culture with ALK5 inhibitor II in an adipocyte induction medium. Particularly, in a group cultured in an adipocyte induction medium added with ALK5 inhibitor II for 0-8 days and thereafter cultured for 6 days in an adipocyte induction medium free of ALK5 inhibitor II (group 6), the highest expression of UCP1 gene was induced and therefore it is clear that fibroblasts were most strongly induced into brown adipocytes. High expression of UCP1 gene was also induced under other conditions in which culturing was performed in the presence of ALK5 inhibitor II (groups 3-5, 7, 8).

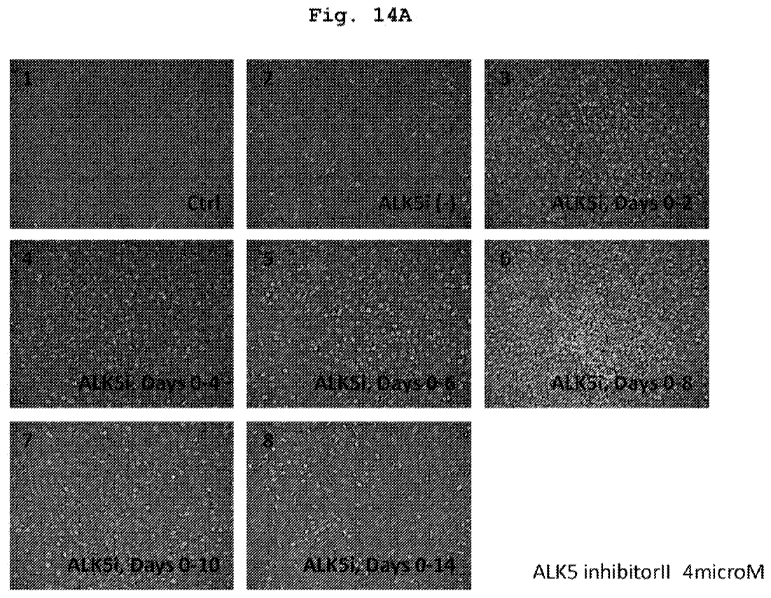

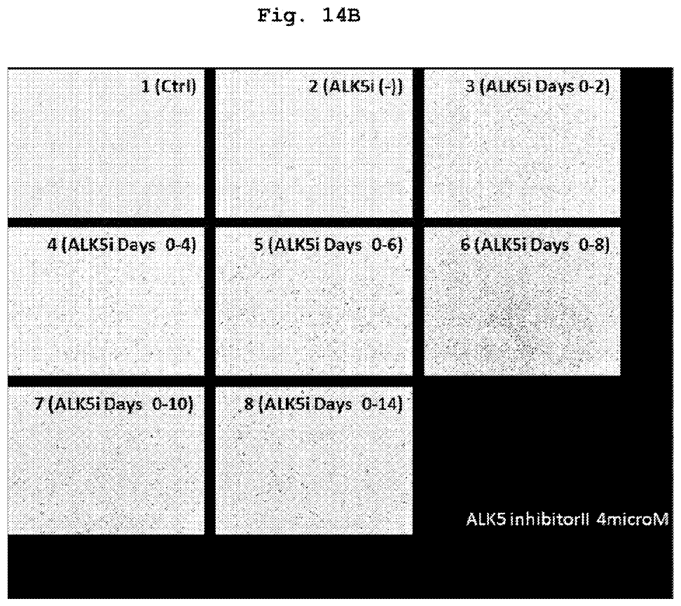

### Example 14 (FIG. 14)

Human normal skin-derived fibroblast (human dermal fibroblasts; HDFs) were suspended in a normal medium (10% FBS-added Dulbecco's modified minimum essential medium; DMEM). This was seeded in a 12-well plate at a concentration of 3×10⁴ cells/well and culturing was started at 5% CO₂/95% humidified air, 37° C. The next day (day 0), the culture supernatant was removed by suction and a normal medium (group 1), an adipocyte induction medium (group 2), or an adipocyte induction medium (groups 3-8) added with ALK5 inhibitor II at concentration of 4 μM was added at 1 mL/well.

The adipocyte induction medium is DMEM added with 1 nM T3, 1 μM Rosiglitazone, 0.5 mM isobutylmethylxanthine (IBMX), 0.5 μM dexamethason, 1 μg/mL Insulin and 10% EBS.

Once every 2 days, the medium was substituted by a fresh one. In groups 3-7, the cells were cultured in an adipocyte induction medium added with ALK5 inhibitor II only in the periods of Days 0-2, Days 0-4, Days 0-6, Days 0-8 and Days 0-10, respectively, and thereafter cultured in an adipocyte induction medium without addition of ALK5 inhibitor II. In group 8, the cells were cultured in an adipocyte induction medium added with ALK5 inhibitor II throughout the whole period of Days 0-14. On day 14, the culture medium was removed from each well by suction, and the cells were washed with PBS(−). On day 14, the culture medium was removed from each well by suction, and the cells were washed with PBS(−). The cells were fixed with 4% para-formaldehyde, washed with PBS(−), Perm Buffer (0.2% Triton-X-added PBS) was added and the cells were incubated for 15 min. After washing 3 times with PBS(−), Blocking One was added and the cells were incubated at room temperature for 60 min. An anti-UCP-1 antibody (RD MAB6158) was added and the mixture was reacted at room temperature for 2 hr and washed 3 times with Wash buffer. CF488-conjugated anti-mouse Ig antibody (Biotum 20014) was added and the mixture was reacted at room temperature for 2 hr and washed 3 times with PBS(−). The cells were subjected to nuclear staining with SlowFade Gold antifade reagent with DAPI manufactured by Life Technologies and photographed at a magnification of 100 with a fluorescence microscope.

The results are shown in FIGS. 14A and 14B (fluorescence microscopic images). It is clear that fibroblasts were converted to brown adipocytes highly expressing UCP1 protein in the group added with ALK5 inhibitor II. Particularly, in a group cultured in an adipocyte induction medium added with ALK5 inhibitor II for 0-8 days and thereafter cultured for 6 days in an adipocyte induction medium free of ALK5 inhibitor II (in FIG. #6), the staining intensity of UCP1 protein was high and many staining positive cells were present and therefore it is clear that fibroblasts were most strongly induced into brown adipocytes. High expression of UCP1 protein was also detected under other conditions in which culturing was performed in the presence of ALK5 inhibitor II (in FIG. #2-5, 7, 8).

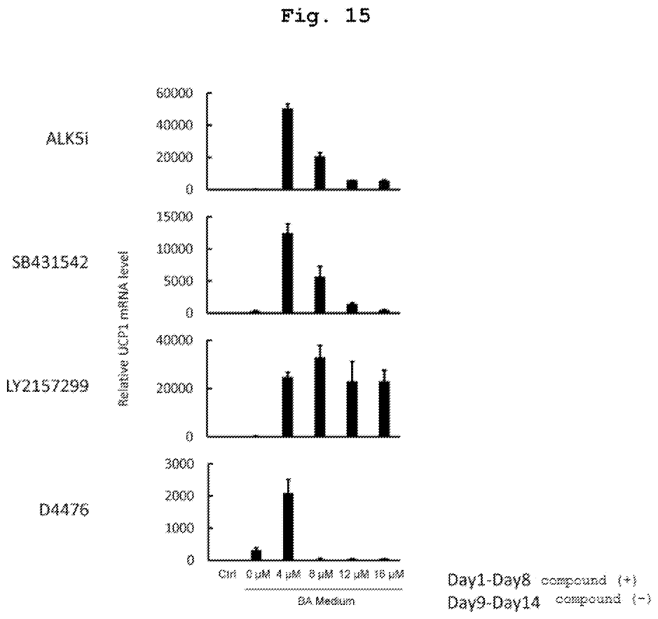

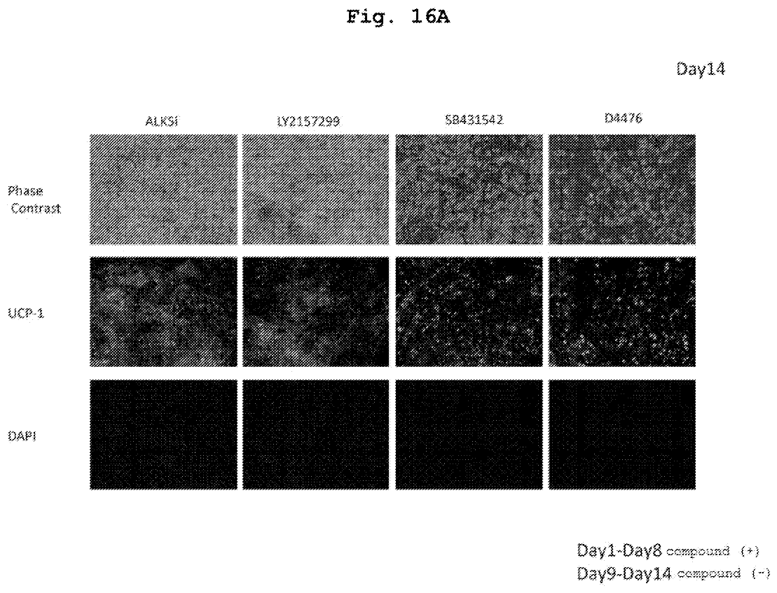

### Example 15 (FIG. 15)

Human normal skin-derived fibroblast (human dermal fibroblasts; HDFs) were suspended in a normal medium (10% FBS-added Dulbecco's modified minimum essential medium; DMEM). This was seeded in a 12-well plate at a concentration of 3×10⁴ cells/well and culturing was started at 5% CO₂/95% humidified air, 37° C. (Day −1). In the control (Ctrl) group, the culture supernatant of the next day (Day 0) was removed by suction and the cells were cultured in a normal medium up to day 14 while substituting the medium with a fresh one once every other day. In groups other than the control (Ctrl) group, the culture supernatant was removed by suction on day 0, an adipocyte induction medium or an adipocyte induction medium added with any of ALK5 inhibitor II, SB431542, LY2157299 and D4476 at a concentration of 4 μM, 8 μM, 12 μM or 16 μM respectively was added at 1 mL/well.

The adipocyte induction medium is DMEM added with 1 nM T3, 1 μM Rosiglitazone, 0.5 mM isobutylmethylxanthine (IBMX), 0.5 μM dexamethason, 1 μg/mL Insulin and 10% FBS.

Once every 2 days, the medium was substituted by a fresh one and the cells were cultured up to Day 9. Thereafter, the cells were cultured in an adipocyte induction medium free of any compound of ALK5 inhibitor II, SB431542, LY215799 and D4476 during Day 9-Day 14. On day 14, the medium was removed by suction from the wells of all groups, the cells were washed with PBS(−) and total RNA was extracted from the cells by using RNA easy Mini Kit manufactured by Qiagen. cDNA was synthesized from the RNA by using Rever Tra Ace qPCR RT Master Mix. The cDNA was admixed with Real-time PCR Master Mix, primers specific to UCP1 gene or β actin gene and Tacpan probe. qRT-PCR was performed using AB7300 Real-time PCR system. The mRNA level of UCP1 gene was quantified as a ratio to β actin gene mRNA and calculated with the value of fibroblast cultured in the normal medium as 1.

The results are shown in FIG. 15. It is clear that fibroblasts were converted to brown adipocytes strongly expressing UCP1 gene by culturing with the addition of any of ALK5 inhibitor II, SB431541, LY2157299 and D4476. Particularly, it is clear that fibroblasts were most strongly induced into brown adipocytes by ALK5 inhibitor II, and LY2157299 was second most strong. In this Example, induction efficiency into brown adipocytes was in the order of ALK5 inhibitor II>LY2157299>SB431541>D4476.

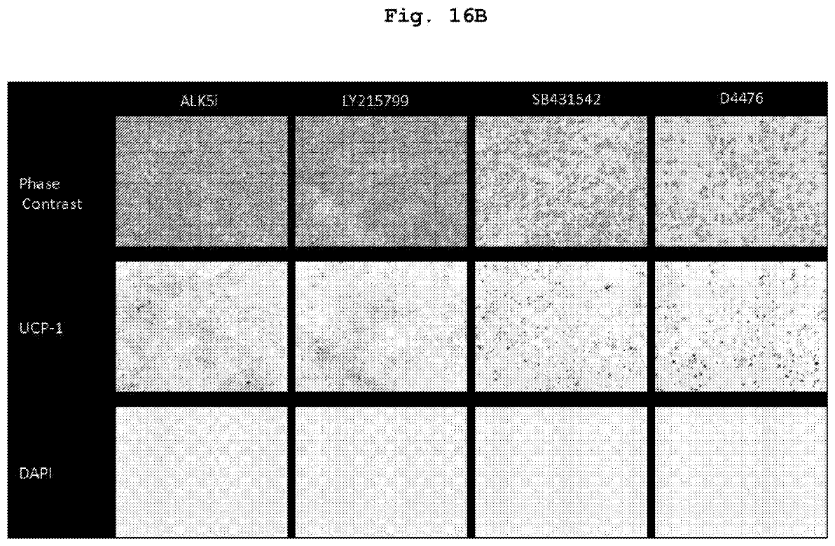

### Example 16 (FIG. 16)

Human normal skin-derived fibroblast (human dermal fibroblasts; HDFs) were suspended in a normal medium (10% FBS-added Dulbecco's modified minimum essential medium added with; DMEM). This was seeded in a 12-well plate at a concentration of 3×10⁴ cells/well and culturing was started at 5% CO₂/95% humidified air, 37° C. The next day (Day 0), the culture supernatant was removed by suction and an adipocyte induction medium added with any compound of ALK5 inhibitor II (4 μM), LY2157299 (8 μM), SB431542 (4 μM) and D4476 (4 μM) was added at 1 mL/well.

The adipocyte induction medium is DMEM added with 1 nM T3, 1 μM Rosiglitazone, 0.5 mM isobutylmethylxanthine (IBMX), 0.5 μM dexamethason, 1 μg/mL Insulin and 10% FBS.

Once every 2 days, the medium was substituted by a fresh one and the cells were cultured up to Day 9. Thereafter, the cells were cultured in an adipocyte induction medium free of any compound of ALK5 inhibitor II, SB431542, LY215799 and D4476 during Day 9-Day 14. On day 14, the medium was removed by suction from each well, and the cells were washed with PBS(−). The cells were fixed with 4% para-formaldehyde, washed with PBS(−), Perm Buffer (0.2% Triton-X-added PBS) was added and the cells were incubated for 15 min. After washing 3 times with PBS(−), Blocking One was added and the cells were incubated at room temperature for 60 min. An anti-UCP-1 antibody (RD MAB6158) was added and the mixture was reacted at room temperature for 2 hr and washed 3 times with Wash buffer. CF488-conjugated anti-mouse Ig antibody (Biotum 20014) was added and the mixture was reacted at room temperature for 2 hr and washed 3 times with PBS(−). The cells were subjected to nuclear staining with SlowFade Gold antifade reagent with DAPI manufactured by Life Technologies and photographed at a magnification of 100 with a fluorescence microscope.

The results thereof are shown in FIG. 16A and FIG. 16B (fluorescence microscopic images). It is clear that fibroblasts were induced into brown adipocytes expressing UCP1 protein by the addition culture with any of ALK5 inhibitor II, LY2157299, SB431541 and D4476. Particularly, it is clear that expression of UCP1 gene was most strongly induced by AKL5 inhibitor II, and LY2157299 was second most strong.

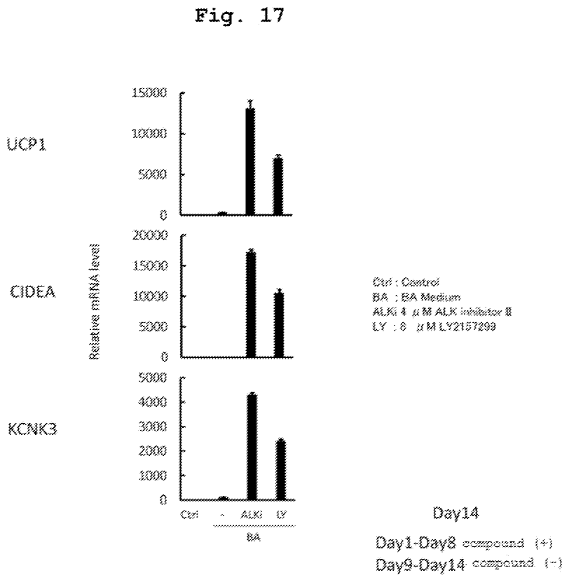

### Example 17 (FIG. 17)

Human normal skin-derived fibroblast (human dermal fibroblasts; HDFs) were suspended in a normal medium (10% FBS-Dulbecco's modified minimum essential medium; DMEM). This was seeded in a 12-well plate at a concentration of 3×10⁴ cells/well and culturing was started at 5% CO₂/95% humidified air, 37° C. (Day −1). In the control (Ctrl) group, the culture supernatant of the next day (Day 0) was removed by suction and the cells were cultured in a normal medium up to day 14 while substituting the medium with a fresh one once every other day. In groups other than the control (Ctrl) group, the culture supernatant was removed by suction on day 0, and an adipocyte induction medium added with any of ALK5 inhibitor II (4 μM) and LY2157299 (8 μM) was added at 1 mL/well.

The adipocyte induction medium is DMEM added with 1 nM T3, 1 μM Rosiglitazone, 0.5 mM isobutylmethylxanthine (IBMX), 0.5 μM dexamethason, 1 μg/mL Insulin and 10% FBS.

Once every 2 days, the medium was substituted by a fresh one and the cells were cultured up to Day 9. Thereafter, the cells were cultured in an adipocyte induction medium free of any compound of ALK5 inhibitor II and LY215799 during Day 9-Day 14. On day 14, the medium was removed by suction from the wells of all groups, and the cells were washed with PBS(−). Total RNA was extracted from the cells by using RNA easy Mini Kit manufactured by Qiagen. cDNA was synthesized from the RNA by using Rever Tra Ace qPCR RT Master Mix. The cDNA was admixed with Real-time PCR Master Mix, primers specific to UCP1 gene, CIDEA gene, KCNK3 gene or β actin gene and Tagman probe. qRT-PCR was performed using AB7300 Real-time PCR system. The mRNA level of UCP1 gene was quantified as a ratio to a actin gene mRNA and calculated with the value of fibroblast cultured in the normal medium as 1.

The results are shown in FIG. 17. It is clear that fibroblasts were induced into brown adipocytes expressing UCP1 gene, CIDEA gene and KCNK3 gene by the addition culture with any of ALK5 inhibitor II and LY2157299. Particularly, it is clear that expression of UCP1 gene was more strongly induced by AKL5 inhibitor II.

1-9. (canceled)

10. A method for generating a brown adipocyte, comprising:
culturing a fibroblast in a medium in the presence of a TGFβ/SMAD pathway inhibitor selected from the group consisting of an ALK4 inhibitor, an ALK5 inhibitor, and an ALK7 inhibitor,
wherein the medium comprises insulin, 3-isobutyl-1-methylxanthine, and dexamethasone.

11. A kit for converting a fibroblast to a brown adipocyte, comprising:
a TGFβ/SMAD pathway inhibitor selected from the group consisting of an ALK4 inhibitor, an ALK5 inhibitor, and an ALK7 inhibitor, and
a medium comprising insulin, 3-isobutyl-1-methylxanthine, and dexamethasone.

12. The method according to claim 10, wherein the TGFβ/SMAD pathway inhibitor is selected from the group consisting of D4476, SB431542, and ALK-5 inhibitor II.

13. The kit according to claim 11, wherein the TGFβ/SMAD pathway inhibitor is selected from the group consisting of D4476, SB431542, and ALK-5 inhibitor II.

14. The method according to claim 10, wherein the medium further comprises indomethacin.

15. The kit according to claim 11, wherein the medium further comprises indomethacin.

16. The method according to claim 10, wherein the medium further comprises T3 and rosiglitazone.

17. The kit according to claim 11, wherein the medium further comprises T3 and rosiglitazone.

18. The method according to claim 12, wherein the medium further comprises T3 and rosiglitazone.

19. The kit according to claim 13, wherein the medium further comprises T3 and rosiglitazone.
